SEQUENCE LISTING (11 sequences)

SEQ ID NO: 1 - protein, 497 aa, Alphainfluenzavirus influenzae
ATKGTKRSYEQMETDGERQNATEIRASVGKMIDGIGRFYIQMCTELKLSDYEGRLIQNSLTIERMVLSAFDERRNKYLEEHPSAGKDPKKTGGPIYRRVDGKWRRELILYDKEEIRRIWRQANNGDDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRTMVDQVRESRNPGNAEFEDLIFLARSALILRGSVAHKSCLPACVYGSAVASGYDFEREGYSLVGIDPFRLLQNSQVYSLIRPNENPAHKSQLVWMACHSAAFEDLRVSSFIRGTKVVPRGKLSTRGVQIASNENMETMESSTLELRSRYWAIRTRSGGNTNQQRASSGQISIQPTFSVQRNLPFDRPTIMAAFTGNTEGRTSDMRTEIIRLMESARPEDVSFQGRGVFELSDEKATSPIVPSFDMSNEGSYFFGDNAEEYDN

SEQ ID NO: 2 - protein, 48 aa, synthetic construct
  REGION 1..48: note = hybrid human chicken C4Bp
KKQGDADVCGEVAYIQSVVSDCHVPTAELRTLLEIRKLFLEIQKLKVE

SEQ ID NO: 3 - skipped

SEQ ID NO: 4 - protein, 7 aa, synthetic construct
  REGION 1..7: note = Postively charged peptide
GRRRRRS

SEQ ID NO: 5 - protein, 55 aa, synthetic construct
  REGION 1..55: note = OVX313
KKQGDADVCGEVAYIQSVVSDCHVPTAELRTLLEIRKLFLEIQKLKVEGRRRRRS

SEQ ID NO: 6 - protein, 554 aa, synthetic construct
  REGION 1..554: note = OVX836
ATKGTKRSYEQMETDGERQNATEIRASVGKMIDGIGRFYIQMCTELKLSDYEGRLIQNSLTIERMVLSAFDERRNKYLEEHPSAGKDPKKTGGPIYRRVDGKWRRELILYDKEEIRRIWRQANNGDDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRTMVDQVRESRNPGNAEFEDLIFLARSALILRGSVAHKSCLPACVYGSAVASGYDFEREGYSLVGIDPFRLLQNSQVYSLIRPNENPAHKSQLVWMACHSAAFEDLRVSSFIRGTKVVPRGKLSTRGVQIASNENMETMESSTLELRSRYWAIRTRSGGNTNQQRASSGQISIQPTFSVQRNLPFDRPTIMAAFTGNTEGRTSDMRTEIIRLMESARPEDVSFQGRGVFELSDEKATSPIVPSFDMSNEGSYFFGDNAEEYDNGSKKQGDADVCGEVAYIQSVVSDCHVPTAELRTLLEIRKLFLEIQKLKVEGRRRRRS

SEQ ID NO: 7 - DNA, 1491 bp, Alphainfluenzavirus influenzae
  source 1..1491: mol_type = genomic DNA
gcgactaagggcacgaaacgcagctacgaacaaatggaaaccgacggtgagcgtcaaaatgcaaccgaaatccgcgctagcgtcggcaagatgatcgacggcatcggccgtttttacattcagatgtgcaccgagctgaagctgagcgattacgagggtcgtctgattcagaatagcttgacgatcgagcgtatggtgttgagcgcgttcgatgagcgccgcaacaaatatctggaagaacatccgagcgccggtaaagatccgaagaaaaccggtggccctatctaccgtcgtgttgatggcaagtggcgtcgcgagctgattctgtatgacaaagaagaaattcgccgtatttggcgccaggcgaataatggtgacgacgcgaccgcgggtttaacgcacatgatgatctggcattccaacctgaacgatgcgacgtatcaacgtacccgtgcgctggtgcgtaccggcatggacccacgtatgtgctcgctgatgcaaggttccaccctgcctcgtcgtagcggtgctgccggtgcggcagtgaaaggtgtcggcacgatggtcatggaacttatccgcatgattaagcgcggtatcaatgatcgtaatttctggcgcggtgagaatggtcgtcgtacccgtattgcgtatgagcgtatgtgcaacattctgaagggtaaattccagaccgcggcacagcgtacgatggtcgaccaagttcgcgagtctcgtaacccgggcaatgctgagtttgaagatctgattttcctggcgcgtagcgccctgattctgcgtggctcggttgcgcacaaatcttgtctgccggcctgcgtctatggtagcgcggtggcatccggttacgactttgagcgtgagggttatagcttggttggcattgacccgtttcgcctgctgcagaacagccaggtgtacagcctgatccgtccaaatgagaacccggcacacaagtcccaactggtttggatggcatgtcatagcgcggctttcgaagatctgcgtgtgtctagctttatccgcggtaccaaagttgtgccgcgtggcaagctgagcacgcgtggtgtgcaaatcgccagcaacgaaaacatggaaaccatggaatcttcaaccctggagctgcgtagccgttactgggcgattcgcacccgcagcggtggcaataccaaccagcaacgtgcgagcagcggccagatcagcattcaaccgacttttagcgttcagcgtaatctgccgttcgaccgcccgacgatcatggcagcctttaccggtaacaccgagggtcgcactagcgacatgcgcaccgaaatcattcgcctgatggagagcgcccgtccggaagatgtcagcttccagggtcgtggtgttttcgagctgagcgacgagaaagcgacctccccgatcgtcccgagctttgacatgtctaacgagggcagctactttttcggtgataatgcagaagagtacgataac

SEQ ID NO: 8 - DNA, 144 bp, synthetic construct
  misc_feature 1..144: note = Hybrid human chicken C4Bp
  source 1..144: mol_type = other DNA
aagaaacagggtgatgctgacgtgtgcggcgaagtggcatatatccagagcgtcgtgagcgattgtcacgttccgacggcagagttgcgcacgctgttggaaatccgtaagctgttcttggagattcaaaagctcaaagttgag

SEQ ID NO: 9 - DNA, 21 bp, synthetic construct
  misc_feature 1..21: note = Postively charged peptide
  source 1..21: mol_type = other DNA
ggtcgtcgtcgcagacgttcc

SEQ ID NO: 10 - DNA, 165 bp, synthetic construct
  misc_feature 1..165: note = OVX313
  source 1..165: mol_type = other DNA
aagaaacagggtgatgctgacgtgtgcggcgaagtggcatatatccagagcgtcgtgagcgattgtcacgttccgacggcagagttgcgcacgctgttggaaatccgtaagctgttcttggagattcaaaagctcaaagttgagggtcgtcgtcgcagacgttcc

SEQ ID NO: 11 - DNA, 1662 bp, synthetic construct
  misc_feature 1..1662: note = OVX836
  source 1..1662: mol_type = other DNA
gcgactaagggcacgaaacgcagctacgaacaaatggaaaccgacggtgagcgtcaaaatgcaaccgaaatccgcgctagcgtcggcaagatgatcgacggcatcggccgtttttacattcagatgtgcaccgagctgaagctgagcgattacgagggtcgtctgattcagaatagcttgacgatcgagcgtatggtgttgagcgcgttcgatgagcgccgcaacaaatatctggaagaacatccgagcgccggtaaagatccgaagaaaaccggtggccctatctaccgtcgtgttgatggcaagtggcgtcgcgagctgattctgtatgacaaagaagaaattcgccgtatttggcgccaggcgaataatggtgacgacgcgaccgcgggtttaacgcacatgatgatctggcattccaacctgaacgatgcgacgtatcaacgtacccgtgcgctggtgcgtaccggcatggacccacgtatgtgctcgctgatgcaaggttccaccctgcctcgtcgtagcggtgctgccggtgcggcagtgaaaggtgtcggcacgatggtcatggaacttatccgcatgattaagcgcggtatcaatgatcgtaatttctggcgcggtgagaatggtcgtcgtacccgtattgcgtatgagcgtatgtgcaacattctgaagggtaaattccagaccgcggcacagcgtacgatggtcgaccaagttcgcgagtctcgtaacccgggcaatgctgagtttgaagatctgattttcctggcgcgtagcgccctgattctgcgtggctcggttgcgcacaaatcttgtctgccggcctgcgtctatggtagcgcggtggcatccggttacgactttgagcgtgagggttatagcttggttggcattgacccgtttcgcctgctgcagaacagccaggtgtacagcctgatccgtccaaatgagaacccggcacacaagtcccaactggtttggatggcatgtcatagcgcggctttcgaagatctgcgtgtgtctagctttatccgcggtaccaaagttgtgccgcgtggcaagctgagcacgcgtggtgtgcaaatcgccagcaacgaaaacatggaaaccatggaatcttcaaccctggagctgcgtagccgttactgggcgattcgcacccgcagcggtggcaataccaaccagcaacgtgcgagcagcggccagatcagcattcaaccgacttttagcgttcagcgtaatctgccgttcgaccgcccgacgatcatggcagcctttaccggtaacaccgagggtcgcactagcgacatgcgcaccgaaatcattcgcctgatggagagcgcccgtccggaagatgtcagcttccagggtcgtggtgttttcgagctgagcgacgagaaagcgacctccccgatcgtcccgagctttgacatgtctaacgagggcagctactttttcggtgataatgcagaagagtacgataacggcagcaagaaacagggtgatgctgacgtgtgcggcgaagtggcatatatccagagcgtcgtgagcgattgtcacgttccgacggcagagttgcgcacgctgttggaaatccgtaagctgttcttggagattcaaaagctcaaagttgagggtcgtcgtcgcagacgttcc

(19) United States
(12) Patent Application Publication
(10) Pub. No.: US 2024/0398928 A1
(43) Pub. Date: Dec. 5, 2024

(54) IMMUNOGENIC COMPOSITIONS AND THEIR USE

(71) Applicants: OSIVAX, Paris (FR); OSIVAX Belgique, Liege (BE)

(72) Inventors: Alexandre LE VERT, Paris (FR); Julien BOULEY, Apprieu (FR); Florence NICOLAS, Genas (FR); Paul WILLEMS, Jodoigne (BE); Delphine GUYON-GELLIN, Saint-Cyr-au-Mont-d'Or (FR)

(21) Appl. No.: 18/686,381

(22) PCT Filed: Aug. 24, 2022
(86) PCT No.: PCT/EP2022/073630

§ 371 Date: Feb. 23, 2024

(30) Foreign Application Priority Data
Aug. 24, 2021 (EP) 21306142.7
Sep. 15, 2021 (EP) 21196968.8
Dec. 7, 2021 (EP) 21212974.6
Mar. 14, 2022 (EP) 22161930.7
May 2, 2022 (EP) 22305655.7
Jul. 28, 2022 (EP) 22306128.4

(51) Int. Cl. A61K 39/145; A61K 39/00; C07K 14/005

(52) U.S. Cl. CPC A61K 39/145; C07K 14/005; A61K 2039/54; A61K 2039/545; C07K 2319/00; C12N 2760/16122; C12N 2760/16134

(57) ABSTRACT

The invention relates to immunogenic compositions and their use as a vaccine for the prevention of influenza disease in a human subject. More specifically, the invention relates to methods of use of an immunogenic composition as a vaccine or immunotherapy in the prevention or treatment of influenza disease in a human subject in need thereof, said immunogenic composition comprising: a fusion protein comprising (i) an influenza nucleoprotein antigen and, (ii) a carrier protein comprising a self-assembling polypeptide derived from C4bp oligomerization domain and a positively charged tail, wherein an amount of 180 μg, or more, of said fusion protein is administered to said human subject.

### CROSS REFERENCE TO RELATED APPLICATIONS

This application is a United States Application under 35 U.S.C. 371 claiming benefits of PCT Application No. PCT/EP2022/073630, filed on Aug. 24, 2022, which claims the benefit of priority from European Patent Application No. 22306128.4, filed on Jul. 28, 2022, European Patent Application No. 22305655.7, filed on May 2, 2022, European Patent Application No. 22161930.7, filed on Mar. 14, 2022, European Patent Application No. 21212974.6, filed on Dec. 7, 2021, European Patent Application No. 21196968.8, filed on Sep. 15, 2021, and European Patent Application No. 21306142.7, filed on Aug. 24, 2021, the contents of each of which are incorporated herein by reference.

### REFERENCE TO AN ELECTRONIC SEQUENCE LISTING

The contents of the electronic sequence listing (OVX-02-US-Sequence Listing.xml; Size: 17,487 bytes; and Date of Creation: Feb. 22, 2024, is herein incorporated by reference in its entirety.

### TECHNICAL DOMAIN

The disclosure relates to immunogenic compositions and their use as a vaccine for the prevention of influenza disease in a human subject.

### BACKGROUND

Seasonal influenza is estimated to result in about 3 to 5 million cases of severe illness worldwide every year, with about 290,000 to 650,000 deaths, mostly among people aged 65 years or older [Iuliano A D, et al. Lancet. 2018; 391:1285-1300].

Annual vaccination is considered the most effective way to prevent influenza. Among the general population in the USA, seasonal trivalent or quadrivalent influenza vaccine effectiveness has been limited to 42% on average over the last ten years (32% in the elderly) [CDC-Past Seasons Vaccine Effectiveness Estimates. Available from: https://www.cdc.gov/flu/vaccines-work/past-seasons-estimates.html. Accessed 21 May 2021]. Similar data are available in Europe [Krammer F, et al. Influ Respir Viruses. 2020; 14:237-243]. When circulating viruses did not match the vaccine viruses, effectiveness may drop to 10-20% only, as in the season 2014-2015. There is therefore a medical need for improving influenza vaccines efficacy.

Although antibody threshold values for viral surface hemagglutinin (HA) and neuraminidase (NA) are recognized as surrogates/correlates for efficacy in clinical trials on most current vaccines, cellular responses, in particular CD4- and CD8-mediated responses, are very likely to contribute to protection, in particular in the elderly population [McElhaney J E, et al. Front Immunol. 2016; 7:41. Trombetta C M, et al. Expert Rev Vaccines. 2016; 15:967-976. Pleguezuelos O, et al. Clin Vaccine Immunol. 2015; 22:949-956; Savic M, et al. Immunology. 2016; 147:165-177].

In an attempt to improve over currently available influenza vaccines through a T-cell response, the virus nucleoprotein (NP) appears as a target of choice. This internal protein is highly conserved across A strains, as well as between A and B strains, and provides structural and functional support to the viral replication machinery [Ye Q, Krug R M, Tao Y J. Nature. 2006; 444:1078-1082]. In humans, there is growing evidence on the role of T-cell immunity against conserved internal antigens in the protection against influenza. A prospective cohort study conducted during the H1N1 pandemic of 2009 showed that higher frequencies of pre-existing T-cells specific to conserved CD8 epitopes were found in individuals who developed less severe illness [Sridhar S, et al. Nat Med. 2013; 19:1305-1312]. The Flu Watch Cohort Study has suggested that pre-existing T-cell responses targeting internal viral proteins provides protective immunity against pandemic and seasonal influenza. The presence of NP-specific T-cells (above a threshold of 20 Spot Forming Unit [SFC]/million peripheral blood mononuclear cells [PBMC]) before exposure to virus correlated with fewer cases of symptomatic, polymerase chain reaction (PCR)-positive influenza A, during both pandemic and seasonal influenza periods [Hayward A C, et al. Am J Respir Crit Care Med. 2015; 191:1422-1431.]. These results provide the rationale to develop NP-based vaccines against influenza.

OVX836 (OSIVAX, Lyon, France) is a recombinant protein developed as a broad-spectrum vaccine against all influenza strains. The antigenic part corresponds to the NP sequence of the A/WSN/1933 (H1N1) influenza virus. OVX836 protein contains 7 copies of NP, each fused to OVX313. The OVX313 sequence is derived from the C-terminal oligomerization domain of the human C4b binding protein (hC4BP) [Hofmeyer T, et al. J Mol Biol. 2013; 425:1302-1317], but modified to minimize homology with the human sequence (hybrid chicken sequence; homology less than 20%). When fused by deoxyribonucleic acid (DNA) engineering to an antigen, and after protein expression, OVX313 has the unique property to heptamerize antigens, thus improving the antigen's accessibility to the immune system and increasing their humoral and cellular immune responses [Del Campo J., et al. npj Vaccines. 2019; 4:4]. As NP is not subject to antigenic variation, OVX836 would not have to be adapted annually, as required for current seasonal influenza vaccines. Animal studies have demonstrated OVX836's ability to elicit humoral and cellular immunity—including CD8+ T-cells in the lungs—as well as protection in mice [Del Campo J., et al. npj Vaccines. 2019; 4:4] and ferrets [Del Campo J, et al. Options X Control Influenza-Singapore 2019; Abstract No 10936:456] against influenza challenges. Importantly, OVX836 protected mice against viral challenge with three different influenza A subtypes isolated several decades apart, and this was accompanied by a reduction in viral load. Both CD4+ and CD8+ T-cells might be involved in infected cells destruction, although recent nonclinical experiments with OVX836 in mice support CD8+ T-cells as the most effective immune response [Del Campo J, et al. Front Immunol. 2021. https://www.frontiersin.org/articles/10.3389/fimmu.2021.678483/abstract. Accessed 21 May 2021].

A first-in-human clinical study was performed to assess the safety and immunogenicity of OVX836.

There is still a need to further improve the dosage regimen and formulation of an immunogenic composition comprising OVX836 fusion protein or their functional variants.

### BRIEF DESCRIPTION

One aspect of the present disclosure relates to an immunogenic composition for use as a vaccine or immunotherapy in the prevention or treatment of influenza disease in a human subject in need thereof, said immunogenic composition comprising: a fusion protein comprising
(i) an influenza nucleoprotein antigen and,
(ii) a carrier protein comprising a self-assembling polypeptide derived from C4bp oligomerization domain and a positively charged tail,
   wherein an amount of 180 μg, or more, of said fusion protein is administered to said human subject, for example, an amount comprised between 180 μg and 1000 μg.

Another aspect of the present disclosure is directed to an immunogenic composition comprising a fusion protein as above-defined, at a concentration of 300 μg/mL or above, and one or more pharmaceutically acceptable excipients, in particular for use as a vaccine or immunotherapy in the prevention or treatment of influenza disease in a human subject in need thereof.

### LEGENDS OF THE FIGURES

FIG. 1: Mean, median and SD of the Number of NP-specific IFNγ spot forming T-cells (SFC/million PBMC) in the four treatment groups, at baseline (Day 1) before vaccination.

FIG. 2: Mean, median and SD of the Number of NP-specific IFNγ spot forming T-cells (SFC/million PBMC) in the four treatment groups, at Day 8 post-vaccination.

FIG. 3: Mean, median and SD of the Number of NP-specific IFNγ spot forming T-cells (SFC/million PBMC) at each time point in the four treatment groups, at Day 36 post-vaccination (i.e. 8 days after the 2^{nd} vaccination).

FIG. 4: Over-time evolution of the number of NP-specific IFN-γ Spot Forming T-cells (SFC)/10⁶ cells in the pooled placebo and the three OVX836 vaccinated groups (30 μg, 90 μg and 180 μg) from baseline (Day 1, pre-vaccination) to Day 150 (4 months after 2^{nd} administration). Results are presented as arithmetic means±standard errors. *p<0.05, **p<0.01, Dwass, Steel, Critchlow-Fligner's post-hoc tests versus placebo when the Kruskal-Wallis test was significant.

FIG. 5A: Number of NP-specific IFN-γ Spot Forming Cells (SFC)/106 cells in the pooled placebo and three OVX836 vaccinated groups (30 μg, 90 μg and 180 μg) at baseline (Day 1), and 8 days after 1st (Day 8) and 2nd (Day 36) administrations. *p<0.05, **p<0.01, Dwass, Steel, Critchlow-Fligner's tests. D1=pre-vaccination baseline, D8=8 days after 1st vaccination, D36=8 days after 2nd vaccination.

FIG. 5B: Number of SFC/106 cells in the different groups on Day 8. *p<0.05, **p<0.01, Dwass, Steel, Critchlow-Fligner's post-hoc tests as the Kruskal-Wallis test was significant (p=0.002). In both panels, results are presented as box plots showing the median (horizontal bar in the box), the interquartile interval (extremities of the box) and the minimum and maximum values (lower and upper error bars).

FIG. 6A: Over-time evolution of NP-specific immunoglobulin G (IgG) geometric mean titers (GMTs±95% confidence interval [CI]) from baseline (Day 1 pre-vaccination) up to Day 150 (4 months after 2nd administration) in the pooled placebo and three OVX836 vaccine groups (30 μg, 90 μg and 180 μg). *p<0.05; **p<0.01 Dwass, Steel, Critchlow-Fligner's post-hoc tests as the Kruskal-Wallis test was significant.

FIG. 6B: Percentage of subjects presenting a four-fold increase of the NP-specific IgG titer between baseline (Day 1 pre-vaccination) and Day 29 (28 days after 1st administration) in the pooled placebo and three OVX836 vaccine groups (30 μg, 90 μg and 180 μg). *p<0.05; ***p<0.001 Fisher's exact tests.

FIG. 7: Mean Number of NP-specific IFNγ spot forming T-cells (SFC/million PBMC) at each Day 1 and Day 8 in the three treatment groups, in the pooled age strata (Intent-To-Treat (ITT) cohort after elimination of two outlier subjects in the OVX836 180 μg group (subjects 128-095 and 232-365 presenting high baseline values on Day 1:957 and 1630, respectively).

FIG. 8: Percentage of NP-specific CD4+ T cells positive for IFNγ at baseline (Day 1), Day 8, Day 29 and Day 180, in the three treatment groups, in the pooled age strata (Per Protocole Day 29 (PP-D29) cohort).

FIG. 9: Cumulative hazard of non-specific ILIs as a function of time between vaccination and ILI start date during the flu season (2 Dec. 2019 to 9 Mar. 2020)—ITT (Intent-To-Treat Cohort).

FIG. 10: Cumulative hazard of non-specific ILIs, occurring from 14 days post-vaccination, as a function of time between vaccination and ILI start date during the flu season (2 Dec. 2019 to 9 Mar. 2020)—ITT (Intent-To-Treat Cohort).

FIG. 11: Number of ILIs during the flu season (before March 9) and more than 14 days after vaccination—ITT (Intent-To-Treat Cohort).

FIG. 12: Median percentage of NP-specific CD8+ T cells positive for at least IFNγ at Day 1 and Day 8 for the subjects belonging to the lowest quartile of CD8+ response at baseline.

FIG. 13: Over-time evolution of NP-specific immunoglobulin G (IgG) geometric mean titers (GMTs from baseline (Day 1 pre-vaccination) up to Day 29 (1 months after immunisation) in the placebo and three OVX836 vaccine groups (180 μg, 300 μg and 480 μg). ***p<0.001 compared to Placebo; post-hoc Bonferroni's intergroup pairwise comparison as the Anova test was significant.

FIG. 14A: Mean change of NP-specific total T-cell responses evaluated by IFNγ ELISpot at Day 8 vs Day 1 for the placebo group and the three OVX836 vaccine groups (180 μg, 300 μg and 480 μg)—Statistics: Pairwise Fisher's LSD comparison, following confirmation that ANOVA test between groups is significant (p<0.05); Error bars represent the standard error.

FIG. 14B: Mean change of percentage of NP-specific CD4+ T cells positive for IFNγ at Day 8 vs Day 1 for the placebo group and the three OVX836 vaccine groups (180 μg, 300 μg and 480 μg)—Statistics: Pairwise Fisher's LSD comparison, following confirmation that ANOVA test between groups is significant (p<0.05); Error bars represent the standard error.

FIG. 14C: Mean change of percentage of NP-specific CD8+ T cells positive for at least IFNγ at Day 8 vs Day 1 for the placebo group and the three OVX836 vaccine groups (180 μg, 300 μg and 480 μg)—Statistics: Pairwise Fisher's LSD comparison, following confirmation that ANOVA test between groups is significant (p<0.05); Error bars represent the standard error.

FIG. 15: Cumulative hazard of PCR-confirmed ILIs-ITT for the pooled OVX836 groups of the OVX836-003 study (180 μg, 300 μg and 480 μg) and the pooled untreated (FLU-001 study) and placebo groups (OVX836-003 study)—Intent-To-Treat merged Cohorts of the OVX836 and FLU-001 studies.

### DETAILED DESCRIPTION

### Definitions

In order that the present disclosure may be more readily understood, certain terms are first defined. Additional definitions are set forth throughout the detailed description.

The term “amino acid” refers to naturally occurring and unnatural amino acids (also referred to herein as “non-naturally occurring amino acids”), e.g., amino acid analogues and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogues refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogues can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

Substitution refers to the replacement of a naturally occurring amino acid either with another naturally occurring amino acid or with an unnatural amino acid.

As used herein, the term “protein” refers to any organic compounds made of amino acids arranged in one or more linear chains (also referred as “polypeptide”) and folded into a globular form. It includes proteinaceous materials or fusion proteins. The amino acids in such polypeptide chain may be joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation, PASylation and the like, incorporation of non-natural amino acids, amino acid modification for chemical conjugation or other molecule, etc.

The term “recombinant protein”, as used herein, includes proteins that are prepared, expressed, created or isolated by recombinant means, such as fusion proteins isolated from a host cell transformed to express the corresponding protein, e.g., from a transfectoma, etc.

As used herein, the term “fusion protein” refers to a recombinant protein comprising at least one polypeptide chain which is obtained or obtainable by genetic fusion, for example by genetic fusion of at least two gene fragments encoding separate functional domains of distinct proteins. A protein fusion of the present disclosure includes for example at least an influenza nucleoprotein antigen and at least one other moiety, the other moiety being a carrier protein comprising a self-assembling polypeptide derived from C4bp oligomerization domain and a positively charged tail thereof as described below.

As used herein, the term “antigenic” polypeptide includes immunogenic fragments and epitopes of a particular polypeptide (for example the nucleoprotein NP of influenza virus) capable of inducing an immune response against such antigenic polypeptides (for example NP-specific immune response), at least when such antigenic polypeptide is fused to the carrier protein as disclosed herein.

As used herein, the percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the Needleman and Wunsch algorithm (NEEDLEMAN, and Wunsch).

The percent identity between two nucleotide or amino acid sequences may also be determined using for example algorithms such as EMBOSS Needle (pair wise alignment; available at www.ebi.ac.uk, Rice et al 2000 Trends Genet 16:276-277). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

As used herein, the term “subject” includes any human or nonhuman animal. The term “nonhuman animal” preferably includes mammals, such as nonhuman primates, sheep, dogs, cats, horses, etc.

As used herein, a “variant” of a polypeptide may be natural or artificial mutant variants, for example obtained typically by amino acid substitution, deletion or insertion as compared to the corresponding native polypeptide. In certain embodiments, a variant may have a combination of amino acid deletions, insertions or substitutions throughout its sequence, as compared to the parent polypeptide.

In the context of the present disclosure, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

A “functional variant” is a variant which retains the properties of interest of the native polypeptide.

In preferred embodiments, a variant comprises an amino acid sequence which is at least 50%, 60%, 70%, 80%, 90%, or 95% identical to the native polypeptide sequence.

As such, polypeptides containing substitutions, insertions and/or additions, deletions and covalent modifications with respect to reference sequences, in particular the NP fusion proteins disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

### The Influenza Nucleoprotein Antigen

As used herein, the term “influenza nucleoprotein antigen” refers to any natural influenza nucleoprotein or their antigenic variants.

Natural influenza nucleoproteins include the nucleoproteins of any of the three types A, B and C of the Influenza virus, preferably of type A.

In some embodiments, the nucleoprotein antigen (NP) is derived from viral strain of Influenza A or Influenza B or combinations thereof. In some embodiments, the strain of Influenza A or Influenza B is associated with birds, pigs, horses, dogs, humans or non-human primates. In some embodiments, the viral strain is selected from the group consisting of H1N1, H3N2, H7N9, and H10N8.

In specific embodiments, the influenza nucleoprotein antigen is the NP antigen of influenza virus A, more specifically, from strain A/Wilson-Smith/1933 H1N1, comprising the polypeptide of SEQ ID NO:1.

In specific embodiments, an antigenic variant is a fragment of influenza nucleoprotein antigen having at least 50, 100, 150, 200, 250, 300, 350, 400, 450, 490 consecutive amino acid residues of the wild type nucleoprotein of influenza virus A, B or C, preferably derived from SEQ ID NO:1. A fragment of influenza nucleoprotein antigen is by definition at least one amino acid shorter than full length wild-type nucleoprotein of influenza virus A, B or C.

In specific embodiments, an antigenic variant of influenza nucleoprotein antigen is an antigenic polypeptide variant having at least 50%, 60%, 70%, 80%, 90%, 95% or 99% identity to corresponding wild-type sequence of a nucleoprotein of influenza virus A, B, or C. preferably. Preferably, an antigenic variant of influenza nucleoprotein antigen is an antigenic polypeptide variant having at least 50%, 60%, 70%, 80%, 90%, 95% or 99% identity to SEQ ID NO:1.

In a particular embodiment, said variant differs from the corresponding influenza nucleoprotein native antigen, through only amino acid substitutions, with natural or non-natural amino acids, preferably only 1, 2, 3, 4, 5, 6, 7, 8, 9 or 10 amino acid substitutions with natural amino acids, in particular as compared to the native influenza NP antigen of SEQ ID NO:1. In a specific embodiment, a variant is a mutant variant having 1, 2 or 3 amino acid substitutions with natural amino acids as compared to the native influenza NP antigen of SEQ ID NO:1.

In more specific embodiments, the amino acid sequence of said mutant variant may differ from the native influenza NP antigen through mostly conservative amino acid substitutions; for instance at least 10, such as at least 9, 8, 7, 6, 5, 4, 3, 2 or 1 of the substitutions in the variant are conservative amino acid residue replacements.

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also may be substantially retained in a variant mutant polypeptide as compared to a parent polypeptide of any influenza NP antigen, typically of SEQ ID NO: 1.

In specific embodiments, a mutant variant comprises a polypeptide which is identical to SEQ ID NO:1, except for 1, 2 or 3 amino acid residues which have been replaced by another natural amino acid by conservative amino acid substitutions as defined above.

In specific embodiments, a variant of the NP antigen does not comprise any mutation as compared to the parent polypeptide of SEQ ID NO:1 in the epitope recognized by the human immune system as described for example in IEDB database (immune epitope data base) accessible under www.IEDB.org. In specific embodiments, a variant of the NP antigen does not include any mutation as compared to the parent polypeptide of SEQ ID NO:1 in the conserved amino acid residue between NP of strain A and NP of strain B. As used herein, “conserved amino acid residues” correspond to the amino acid residues which are identical between NP of strain A and NP of strain B when aligned using standard sequence protein alignments such as those using BLAST algorithm.

The amino acid residues E₃₃₉ and R₄₁₆ (numbering with N-terminal methionine, M₁) are essential for self-assembling of NP, and not subjected to genetic diversity of influenza A virus. Therefore, in specific embodiments, a variant of the NP antigen comprises E₃₃₉ and R₄₁₆.

### The Carrier Protein

As used herein, the term “carrier protein” designates generally a protein to which antigens are conjugated or fused and thereby rendered more immunogenic. Here the term is used specifically in the meaning of a protein carrying an antigen. The function of the protein is to increase the immunogenicity of said antigen to which it is conjugated or fused.

The carrier protein for use in the fusion protein comprises a self-assembling polypeptide derived from C4bp oligomerization domain and a positively charged tail. The complement inhibitor C4-binding protein (C4bp) is an abundant plasma protein first discovered in mice. Its natural function is to inhibit the classical and lectin pathways of complement activation. The last exon of the C4bp alpha chain gene encodes the only domain in the protein which does not belong to the complement control protein family. This non-complement control protein domain contains 57 amino acid residues in human and 54 amino acid residues in mice and is both necessary and sufficient for the oligomerization of the C4bp. It has been found that, when fused to antigens, said self-assembling polypeptide is also necessary and sufficient for the oligomerization of the resulting fusion protein.

PCT/IB2004/002717 and PCT/EP03/08926 describe the use of mammalian C4bp oligomerization domains to increase the immunogenicity of antigens in mammals. WO2007/062819 further describe a C4bp oligomerization domain of chicken species and variants thereof.

In preferred embodiments, in order to minimize self-immune reaction, the self-assembling polypeptide has an identity to human C4bp which is lower than 30%, preferably lower than 20%.

In particular, in specific embodiments, said self-assembling polypeptide derived from C4bp oligomerization domain comprises or essentially consists of SEQ ID NO:2.

In specific embodiments, a functional variant of the self-assembling polypeptide has at least 50%, 60%, 70%, 80%, 90%, 95% or 99% identity to SEQ ID NO:2.

A functional variant may include any variant with one or more amino acid addition, deletion and/or substitutions as compared to SEQ ID NO:2 which retains the self-assembling property of the polypeptide of SEQ ID NO:2.

In a particular embodiment, said variant differ from SEQ ID NO:2, through only amino acid substitutions, with natural or non-natural amino acids, preferably only 1, 2, 3, 4, 5, 6, 7, 8, 9 or 10 amino acid substitutions with natural amino acids. In a specific embodiment, a variant is a mutant variant having 1, 2 or 3 amino acid substitutions with natural amino acids as compared to SEQ ID NO:2.

In more specific embodiments, the amino acid sequence of said mutant variant may differ from the self-assembling polypeptide of SEQ ID NO:2 through mostly conservative amino acid substitutions; for instance at least 10, such as at least 9, 8, 7, 6, 5, 4, 3, 2 or 1 of the substitutions in the variant are conservative amino acid residue replacements.

The carrier protein further comprises a C-terminal tail consisting of positively charged peptide. The C-terminal tail is preferably a peptide consisting of 6-10 amino acids, with at least 50% of positively charged amino acids. Amino acids with positive charges include arginine or lysine. Examples of such positively charged peptide are disclosed in WO2014/090905 and WO2014/147087.

In preferred embodiments, said positively charged tail comprises the sequence ZXBBBBZ (SEQ ID NO: 3), wherein (i) Z is absent or is any amino acid, (ii) X is any amino acid, and (iii) B is an arginine or a lysine, preferably said positively charged tail comprises or essentially consists of the sequence of SEQ ID NO: 4.

In more preferred embodiments, said carrier protein essentially consists of OVX313 polypeptide, corresponding to the polypeptide of SEQ ID NO:5.

In specific embodiments, said carrier protein is a functional variant of OVX313 polypeptide of SEQ ID NO: 5 having at least 70%, 80%, or more preferably at least 90% identity to SEQ ID NO:5.

In other embodiments, said carrier protein is a functional variant of OVX313 polypeptide of SEQ ID NO:5 which differ from SEQ ID NO:5, by only 1, 2, 3, 4, 5, 6, 7, 8, 9, or 10 amino acids by amino acid substitution. In other embodiments, said carrier protein is a functional variant of OVX313 polypeptide of SEQ ID NO:5 which differ from SEQ ID NO:5, by only 1, 2, 3, 4, 5, 6, 7, 8, 9, or 10 amino acids by conservative amino acid substitution.

### The NP Fusion Protein

The fusion protein for use according to the present disclosure comprises
(i) an influenza nucleoprotein antigen, as defined above, and,
(ii) a carrier protein, as defined above, comprising a self-assembling polypeptide derived from C4bp oligomerization domain and a positively charged tail,

The resulting fusion protein with nucleoprotein antigen is called hereafter for ease of reading the “NP fusion protein”.

In specific embodiments, the carrier protein is fused C-terminally to the nucleoprotein antigen, optionally via a peptide linker. Peptide linker may be any short peptide linker generally used for fusion protein. Preferred peptide linkers, includes glycine-serine linker, such as the dipeptide gly-ser, or gly-ser-ser-ser, or (gly-ser-ser-ser) n, wherein n is an integer between 1 and 4.

In specific embodiments, said NP fusion protein forms heptameric particles after self-assembling.

In specific embodiments, said NP fusion protein form particles with diameters between 15 and 100 nm after self-assembling. The diameter of said particle may be measured for example by dynamic light scattering (DLS). DLS measures the hydrodynamic diameter of particles across the size range of approximatively 0.3 nm to 10 μm. DLS measurements are very sensitive to temperature and dispersant viscosity. Therefore, the temperature must be kept constant at 25° C. and the viscosity of the dispersant must be known.

In specific embodiments, said NP fusion protein form particles with molecular weight between 440 and 2200 kDa.

In more preferred embodiments, said NP fusion protein essentially consists of OVX836 polypeptide, corresponding to the polypeptide of SEQ ID NO:6.

In specific embodiments, said NP fusion protein is a functional variant of OVX836 polypeptide having at least 70%, 80%, or more preferably at least 90% identity to SEQ ID NO:6.

In other embodiments, said NP fusion protein is a functional variant of OVX836 polypeptide of SEQ ID NO: 6 which differ from SEQ ID NO:6, by only 1, 2, 3, 4, 5, 6, 7, 8, 9, or 10 amino acids by amino acid substitution. In other embodiments, said NP fusion protein is a functional variant of OVX836 polypeptide which differ from SEQ ID NO:6, by only 1, 2, 3, 4, 5, 6, 7, 8, 9, or 10 amino acids by conservative amino acid substitution.

### Methods for Preparing the NP Fusion Protein

The NP fusion protein for use according to the present disclosure may be prepared by any conventional methods for preparing recombinant proteins, using nucleic acid molecules that encode said NP fusion protein which nucleotide sequence can be easily derived using the genetic code and, optionally taking into account the codon bias depending on the host cell species.

Examples of nucleotide sequence which can be used to prepare the NP fusion proteins are those encoding the amino acid sequences of SEQ ID NO:1-6, typically as described in Tables 2 and 3.

The nucleic acid molecules may derive from the latter sequences and be optimized for protein expression in prokaryotic cells, for example, in E. coli bacterial cells.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the disclosure can be, for example, DNA or RNA and may or may not contain intronic sequences. In an embodiment, the nucleic acid may be present in a vector such as a recombinant plasmid vector.

Nucleic acids can be obtained using standard molecular biology techniques. Once DNA fragments encoding the nucleoprotein antigen, are obtained, these DNA fragments can be further manipulated by standard recombinant DNA techniques. In these manipulations, a DNA fragment for example encoding the nucleoprotein antigen may be operatively linked to another DNA molecule, for example a fragment encoding the carrier protein and optionally a linker.

The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The NP fusion proteins for use according to the present disclosure (in particular OVX836) can then be produced in a host cell transfectoma using, for example, a combination of recombinant DNA techniques and gene transfection methods as is well known in the art.

For example, to express the NP fusion protein (typically OVX836), corresponding fragments thereof, DNAs encoding partial or full-length recombinant proteins can be obtained by standard molecular biology or biochemistry techniques (e.g., DNA chemical synthesis, PCR amplification or cDNA cloning) and the DNAs can be inserted into expression vectors such that the genes are operatively linked to transcriptional and translational control sequences.

In this context, the term “operatively linked” is intended to mean that a coding polypeptide sequence is ligated into a vector such that transcriptional and translational control sequences within the vector serve their intended function of regulating the transcription and translation of the recombinant NP fusion protein. The expression vector and expression control sequences are chosen to be compatible with the expression host cell used. The protein encoding genes are inserted into the expression vector by standard.

The recombinant expression vector can encode a signal peptide that facilitates secretion of the recombinant fusion protein from a host cell. The NP fusion protein encoding gene can be cloned into the vector such that the signal peptide is linked in frame to the amino terminus of the recombinant protein. The signal peptide can be the native signal peptide of C4bp or a heterologous signal peptide (i.e., a signal peptide from a non-C4bp protein). In specific embodiments, the signal peptide is the methionine amino acid.

In addition to the NP fusion protein encoding sequences, the recombinant expression vectors disclosed herein carry regulatory sequences that control the expression of the recombinant fusion protein in a host cell. The term “regulatory sequence” is intended to include promoters, enhancers and other expression control elements (e.g., polyadenylation signals) that control the transcription or translation of the protein encoding genes. It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1.

In addition to the NP fusion protein encoding sequences and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the NP fusion proteins, the expression vector(s) encoding the recombinant protein is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the proteins of the present disclosure in either prokaryotic or eukaryotic host cells. Expression of the NP proteins may be carried out in prokaryotic cells, for example E. coli host cell. The NP fusion protein may then be recovered by lysis of the bacterial cells, and further purification using standard purification procedures. In specific embodiments, the NP fusion protein is produced according to the method disclosed in DelCampo 2021 (Frontiers in Immunology, doi: 10/3389/fimm.2021.678483.

### Immunogenic Compositions

In another aspect, the present disclosure provides a composition, e.g. an immunogenic composition containing an NP fusion protein as described in the previous sections, at a concentration of 300 μg/mL or above, and one or more pharmaceutically acceptable excipients.

The immunogenic composition includes any aqueous vehicle suitable for a parenteral, intranasal, intramuscular, or subcutaneous administration (e.g., by intramuscular injection). These may be in particular isotonic, sterile, saline solutions (monosodium or disodium phosphate, sodium, potassium, calcium or magnesium chloride and the like or mixtures of such salts).

In specific embodiments, said NP fusion protein comprises at least 400 amino acid residues, for example between 400 and 600 amino acid residues, for example between 540 and 560 amino acid residues, optionally, said NP fusion protein forms particles with diameters between 15 and 100 nm and/or has a molecular weight of between 440 and 2200 kDa in said immunogenic compositions, as disclosed herein.

For example, said immunogenic composition is an aqueous composition which comprises a polypeptide of SEQ ID NO:6 (OVX836) or a variant having at least 70%, 80%, preferably at least 90%, or at least 95% identity to SEQ ID NO:6, at a concentration of 300 μg/mL or above, formulated together with one or more pharmaceutically acceptable excipients.

In specific embodiments, said immunogenic composition may further include one or more of the following excipients such as: a buffer, a salt, an osmolyte, an antioxidant and a surfactant or other agent to prevent protein loss on vial surfaces and/or protein aggregation.

The form of the pharmaceutical compositions, the route of administration, the dosage and the regimen naturally depend upon the condition to be treated, the severity of the illness, the age, weight, and sex of the patient, etc.

For intramuscular administration, for example, the composition is an aqueous solution which should be suitably buffered if necessary and the liquid diluent first rendered isotonic with sufficient saline or glucose. In this connection, sterile aqueous media which can be employed will be known to those of skill in the art in light of the present disclosure. For example, one dosage could be dissolved in 1 ml of isotonic NaCl solution. Examples of formulation for injectable solutions are provided in Remington: The Science and Practice of Pharmacy, 23^{rd} Edition, 2020. Some variation in dosage may occur depending on the condition of the subject being treated.

In specific embodiments, the pH of said composition is between 6.0 and 7.0, preferably between 6.3 and 6.6, for example about 6.5.

In specific embodiments, said immunogenic composition has an osmolality between 300 and 600 mOsm/kg, preferably between 400 and 500 mOsm/kg, for example about 450 mOsm/kg.

In specific embodiments, said immunogenic composition has
(i) a buffering agent for a pH between 6.0 and 7.0, preferably between 6.3 and 6.6, for example about 6.5 and
(ii) an effective amount of osmolytes for an osmolality between 300 and 600 mOsm/kg, preferably between 400 and 500 mOsm/kg, for example about 450 mOsm/kg.

Examples of buffering agent for a pH between 6.0 and 7.0 include sodium citrate or sodium/potassium phosphate buffers.

In specific embodiments, said immunogenic composition further comprises, in addition to the NP fusion protein (typically OVX836), at least
a salt, e.g. sodium sulfate or sodium chloride, preferably sodium sulfate,
an osmolyte, e.g. a sugar such as trehalose or maltose, preferably trehalose,
a buffer, e.g. a phosphate buffer and/or a citrate buffer,
optionally an antioxydant, e.g. methionine,
optionally a surfactant, e.g. polysorbate 80,
   wherein the pH of the composition is between 6.0 and 7.0, typically between 6.3 and 6.6 and the osmolality is between 300 and 600 mOsm/kg.

In specific embodiments, said immunogenic composition further comprises, in addition to the NP fusion protein (typically OVX836), at least
a salt,
trehalose,
a buffering agent for a pH between 6.0 and 7.0, e.g a phosphate buffer and/or a citrate buffer,
optionally an antioxydant, e.g. methionine,
optionally a surfactant, e.g. polysorbate 80,

In more specific embodiments, the immunogenic composition of the present disclosure comprises, in addition to at least the NP fusion protein (typically OVX836):
sodium sulfate or sodium chloride, preferably sodium sulfate,
a sugar, preferably trehalose,
a phosphate buffer and/or a citrate buffer,
optionally an antioxydant, e.g. methionine,
optionally a surfactant, e.g. polysorbate 80,
   wherein the osmolality is between 300 and 600 mOsm/kg, preferably between 400 and 500 mOsm/kg, typically 450 mOsm/kg.

In preferred embodiments, the immunogenic composition of the present disclosure comprises in addition to at least the NP fusion protein (typically OVX836):
sodium sulfate at a concentration of about 75 mM,
trehalose at a concentration of about 200 mM,
optionally, a surfactant such as polysorbate 80 at a concentration between 0.02% and 0.08% (vol/vol), e.g. about 0.04%
optionally an antioxidant such as L-methionine at a concentration of about 5 mM.

In preferred embodiments, the immunogenic composition of the present disclosure comprises in addition to at least the NP fusion protein (typically OVX836):
sodium sulfate at a concentration of about 75 mM,
trehalose at a concentration of about 200 mM,
polysorbate 80 at a concentration between 0.02% and 0.08% (vol/vol), e.g. about 0.04%
L-methionine at a concentration of about 5 mM.

In specific embodiments, said immunogenic composition does not comprise any adjuvant.

In specific embodiments, the immunogenic composition is formulated as a ready-to-use sterile injectable solution.

Sterile injectable solutions are prepared by incorporating the active compound, i.e. the NP fusion protein, in the required amount in the appropriate solvent with various of the other ingredients enumerated above, as required, followed by filtered sterilization.

Methods of Use of the NP Fusion Proteins and their Immunogenic Compositions

The NP fusion proteins (in particular OVX836) and their immunogenic compositions (in particular comprising at least 300 μg/mL of OVX836) as described in the previous sections are useful as a vaccine or immunotherapy in the prevention or treatment of influenza disease in a human subject in need thereof.

Accordingly, the present disclosure provides compositions (e.g., immunogenic compositions as described in the previous section), methods, kits and reagents for prevention and/or treatment of influenza virus in humans and other mammals. The immunogenic compositions disclosed herein can be used as therapeutic or prophylactic agents. They may be used in medicine to prevent and/or treat influenza disease. In exemplary aspects, the immunogenic compositions of the present disclosure are used to provide prophylactic protection from influenza virus. Prophylactic protection from influenza virus can be achieved following administration of an immunogenic composition of the present disclosure, typically with a dose of 180 μg or more of OVX836, 200 μg or more of OVX836, 240 μg or more of OVX836, 300 μg or more of OVX836, or 480 μg or more of OVX836. The immunogenic composition can be administered once, twice, three times, four times or more, preferably as a single dose. It is possible, although less desirable, to administer the immunogenic composition to an infected individual to achieve a therapeutic response. Dosing may need to be adjusted accordingly.

In some embodiments, the immunogenic compositions of the present disclosure can be used as a method of preventing an influenza virus infection in a subject, the method comprising administering to said subject at least one immunogenic composition as provided herein, typically with a dose of 180 μg or more of OVX836, 200 μg or more of OVX836, 240 μg or more of OVX836, 300 μg or more of OVX836, or 480 μg or more of OVX836.

In some embodiments, the immunogenic compositions of the present disclosure can be used as a method of inhibiting a primary influenza virus infection in a subject, the method comprising administering to said subject at least one immunogenic composition as provided herein, typically with a dose of 180 μg or more of OVX836, 200 μg or more of OVX836, 240 μg or more of OVX836, 300 μg or more of OVX836, or 480 μg or more of OVX836. In some embodiments, the immunogenic compositions of the present disclosure can be used as a method of treating an influenza virus infection in a subject, the method comprising administering to said subject at least one immunogenic composition as provided herein, typically with a dose of 180 μg or more of OVX836, 200 μg or more of OVX836, 240 μg or more of OVX836, 300 μg or more of OVX836, or 480 μg or more of OVX836.

In some embodiments, the immunogenic compositions of the present disclosure can be used as a method of reducing an incidence of influenza virus infection in a subject, the method comprising administering to said subject at least immunogenic composition as provided herein typically with a dose of 180 μg or more of OVX836, 200 μg or more of OVX836, 240 μg or more of OVX836, 300 μg or more of OVX836, or 480 μg or more of OVX836.

In some embodiments, the immunogenic composition of the present disclosure can be used as a method of inhibiting spread of influenza virus from a first subject infected with influenza virus to a second subject not infected with influenza virus, the method comprising administering to at least one of said first subject and said second subject at least one immunogenic compositions as provided herein typically with a dose of 180 μg or more of OVX836, 200 μg or more of OVX836, 240 μg or more of OVX836, 300 μg or more of OVX836, or 480 μg or more of OVX836.

Some embodiments of the present disclosure provide methods of inducing an antigen NP specific immune response in a subject, comprising administering to the subject any of the immunogenic compositions as provided herein (preferably an immunogenic composition with OVX836), in an amount effective to produce an NP-specific immune response. In some embodiments, an antigen NP specific immune response comprises total T cell response (in particular CD4 or CD8 NP specific T cell response) or a B cell response (specific anti-NP IgG response).

In some embodiments, a method of producing an antigen NP-specific immune response comprises administering to a subject a single dose of an immunogenic composition of the present disclosure (typically with OVX836, for example a single dose of 180 μg or more, 200 μg or more, 240 μg or more, 300 μg or more, or 480 μg or more).

In some embodiments, the immunogenic composition (typically with OVX836) is administered to a subject by intradermal injection, intramuscular injection, or by intranasal administration. In some embodiments, the immunogenic composition (typically with OVX836) is administered to a subject by intramuscular injection.

In some embodiments, the immunogenic composition is formulated in an effective amount of the NP fusion protein (typically OVX836) to produce an antigen NP-specific immune response in a subject.

The data presented in the Examples demonstrate significant enhanced immune response using the immunogenic compositions as disclosed herein, in particular with OVX836 at a single dose of 180 μg.

In some embodiments, an effective amount of the NP fusion protein (typically OVX836) is a single dose of 180 μg to 1000 μg, 200 μg to 1000 μg, 240 μg to 1000 μg, or 300 μg to 1000 μg, or 480 μg to 1000 μg. In some embodiments, an effective amount of the NP fusion protein (typically OVX836) is a single dose higher than 180 μg administered to the human subject. In some embodiments, an effective amount of the NP fusion protein (typically OVX836) is 200 μg or more administered to the human subject. In some embodiments, an effective amount of the NP fusion protein (typically OVX836) is 240 μg or more administered to the human subject. In some embodiments, an effective amount of the NP fusion protein (typically OVX836) is 300 μg or more administered to the human subject. In some embodiments, an effective amount of the NP fusion protein (typically OVX836) is 480 μg or more administered to the human subject.

In specific embodiments, the immune response may be determined by measuring the increase of NP-specific IFN-γ spot-forming cells (SFCs)/10⁶ PBMCs at least 8 days (day 8 or day 29) after the first injection compared to baseline number of NP-specific IFN-γ spot-forming cells (SFCs)/10⁶ PBMCs at the day of injection (Day 1).

In some embodiments, said subject exhibits at least 50%, 70%, 90%, 110%, 130% increase NP-specific IFN-γ spot-forming cells (SFCs)/10⁶ PBMCs following day 8 of the first dose of the immunogenic composition compared to the baseline (day 1 before injection), for example a first dose of an immunogenic composition comprising 180 μg or more of OVX836.

In specific embodiments, the immune response may be determined either by measuring the increase of NP-specific CD4+ T spot-forming cells (SFCs)/10⁶ PBMCs at least 8 days (day 8 or day 29) after the first injection compared to baseline number at the day of injection (Day 1).

In some embodiments, said subject exhibits at least 100%, 150%, 200%, 250%, 300% or 350% increase NP-specific CD4+ spot-forming cells (SFCs)/10⁶ PBMCs following day 8 of the first dose of the immunogenic composition compared to the baseline (day 1 before injection), for example a first dose of an immunogenic composition comprising 180 μg or more of OVX836.

In some embodiments, said subject exhibits at least 20%, 30%, 50%, 75%, or 100% increase NP-specific CD8+ spot-forming cells (SFCs)/10⁶ PBMCs following day 8 of the first dose of the immunogenic composition compared to the baseline (day 1 before injection), for example a first dose of an immunogenic composition comprising 180 μg or more of OVX836.

The data presented in the Examples also demonstrate significant improved efficacy of the vaccine using the immunogenic compositions as disclosed herein, in particular with OVX836 at a single dose of 180 μg or above which prevents the occurrence of new cases of symptomatic influenza (ILIs) as compared to a single dose of 90 μg which does not protect from symptomatic influenza.

In some embodiments, the immunogenic composition of the present disclosure can be used as a method of providing efficacy against influenza disease, preferably severe influenza, in a subject in need thereof, the method comprising administering to said subject the immunogenic compositions as provided herein (typically with OVX836) with a dose of 180 μg or more, 200 μg or more, 240 μg or more, 300 μg or more, or 480 μg or more.

In some embodiments, the vaccine efficacy may be determined by a significant reduction of the number of influenza like illnesses after 14 days of injection in the patient population treated with the immunogenic compositions of the present disclosure, typically with a dose of 180 μg or more of OVX836, 200 μg or more of OVX836, 240 μg or more of OVX836, 300 μg or more of OVX836, or 480 μg or more of OVX836, as compared to a placebo or to a dose of 90 μg of OVX836.

As used herein, the term “influenza like illness” or “ILI” refers to clinical observation of fever or abrupt onset with more than one of the following symptoms: chills, headache, malaise, myalgia, cough, pharyngitis, and other respiratory complaints.

In some embodiments, a patient population exhibits at least a decrease of 20%, 40%, 60%, 80%, or 95% of influenza like illness after 14 days of injection when treated with the immunogenic compositions of the present disclosure, typically with a dose of 180 μg or more of OVX836 as compared to a patient population receiving a placebo or a dose of 90 μg of OVX836.

In some embodiments, the immunogenic composition of the present disclosure, (typically comprising OVX836) for use as a vaccine protects the subject from severe influenza.

As used herein, the term “severe influenza” refers to the definition of influenza-like illness (ILI; sudden onset of fever and cough or sore throat) and presenting at least one of the following clinical presentations:
Dyspnea, tachypnea, or hypoxia
Radiological signs of lower respiratory tract disease
Central nervous system involvement (e.g., encephalopathy, encephalitis)
Severe dehydration
Acute renal failure
Septic shock
Exacerbation of underlying chronic disease, including asthma, chronic obstructive pulmonary disease (COPD), chronic hepatic or renal insufficiency, diabetes mellitus, or other cardiovascular conditions
Any other influenza-related condition or clinical presentation requiring hospital admission.

In some embodiments, the immunogenic composition of the present disclosure (typically comprising OVX836) for use as a vaccine protects the subject against one or more severe symptoms of severe influenza diseases.

In some embodiments, the immunogenic composition for use as a vaccine immunizes the subject against Influenza for up to 2 years. In some embodiments, the immunogenic composition for use as a vaccine immunizes the subject against Influenza for more than 2 years, more than 3 years, more than 4 years, or for 5-10 years.

In some embodiments, the subject is a young adult between the ages of about 20 years and about 50 years (e.g., about 20, 25, 30, 35, 40, 45 or 50 years old).

In some embodiments, the subject is above 50 years old, for example an elderly subject about 60 years old, about 70 years old, or older (e.g., about 60, 65, 70, 75, 80, 85 or 90 years old).

In some embodiments, the subject has been exposed to influenza; the subject is infected with influenza; or the subject is at risk of infection by influenza.

In other aspects, the disclosure relates to an immunogenic composition for use as a vaccine, or a method of vaccinating a subject comprising administering to the subject an immunogenic composition as disclosed herein, typically comprising OVX836, and more preferably formulated at a concentration of at least 300 μg/mL, wherein a single dose of 180 μg-300 μg, 300 μg-480 μg, or 480 μg-1000 μg, of said NP fusion protein, typically OVX836, is administered to the subject. Preferably, in said method, said immunogenic vaccine is administered by intramuscular injection.

In other aspects, the disclosure relates to the use of a fusion protein as described above, in the preparation of a vaccine for use in the prevention of influenza in a human subject, wherein an amount of 180 μg, or more, of said fusion protein is administered to said human subject, for example, an amount comprised between 180 μg and 1000 μg.

In some embodiments, the immunogenic composition (typically with OVX836) is administered to a subject in combination concomitantly or sequentially, preferably concomitantly, with a second immunogenic composition against influenza comprising one or more inactivated strains of influenza and/or an efficient amount of the hemagglutinin HA antigen from one or more influenza strains. For example, said second immunogenic composition comprises a mixture of inactivated strains of influenza virus strains A and B, for examples a mixture of strains A H1N1, H3N2 and B. In a specific embodiment, said second immunogenic composition is Fluarix.

As used herein, the term “combination”, “combined administration” or “concomitant administration” refers to a combined administration of at least two active ingredients e.g. two immunogenic compositions with distinct antigens or antigenic determinants, where a first immunogenic composition comprising an NP fusion protein as disclosed herein is administered at the same time or separately within time intervals, with a second vaccine or immunogenic composition, in the same subject in need thereof, where these time intervals allow that the combined active ingredients show a cooperative or synergistic effect for the immune response or protection against influenza, typically flu disorder. It is not intended to imply that the immunogenic compositions must be administered at the same time and/or formulated for delivery together although these methods of delivery are within the scope described herein. The terms are also meant to encompass regimens in which the active (immunogenic) agents are not necessarily administered by the same route of administration.

In specific embodiments, one dose of an immunogenic composition of OVX836 of 300 or 480 μg is administered by intramuscular injection concomitantly with one dose of a second immunogenic composition comprising one or more inactivated strains of influenza or influenza hemagglutinin antigens, (e.g. Fluarix vaccine), which may also be administered via intramuscular injection.

The invention will be further illustrated by the following figures and examples. However, these examples and figures should not be interpreted in any way as limiting the scope of the present disclosure.

### SPECIFIC EMBODIMENTS

E1. An immunogenic composition for use as a vaccine or immunotherapy in the prevention or treatment of influenza disease in a human subject in need thereof,
said immunogenic composition comprising: a fusion protein comprising
(i) an influenza nucleoprotein antigen and,
(ii) a carrier protein comprising a self-assembling polypeptide derived from C4bp oligomerization domain and a positively charged tail,
wherein an amount of 180 μg, or more, of said fusion protein is administered to said human subject, for example, an amount comprised between 180 μg and 1000 μg.

E2. The immunogenic composition for use according to Embodiment E1, wherein an amount of 200 μg or more, or 240 μg or more, of said fusion protein is administered to said human subject.

E3. The immunogenic composition for use according to Embodiment E1, wherein an amount of 300 μg or more of said fusion protein is administered to said human subject.

E4. The immunogenic composition for use according to Embodiment E1, wherein an amount of 480 μg or more of said fusion protein is administered to said human subject.

E5. The immunogenic composition for use according to any of the Embodiments E1-E4, wherein the carrier protein is fused C-terminally to the nucleoprotein antigen, optionally via a glycine-serine linker.

E6. The immunogenic composition for use according to any of the Embodiments E1-E5, wherein said fusion protein forms a heptameric particle after self-assembling.

E7. The immunogenic composition for use according to any of the Embodiments E1-E6, wherein said influenza nucleoprotein antigen comprises at least one nucleoprotein antigen from an Influenza strain A, B or C, for example, it essentially consists of the NP antigen of influenza virus A/Wilson-Smith/1933 H1N1.

E8. The immunogenic composition for use according to any of the Embodiments E1-E7, wherein said influenza nucleoprotein antigen comprises
(i) a polypeptide of SEQ ID NO:1, or
(ii) an antigenic polypeptide variant having at least 90% identity to SEQ ID NO:1.

E9. The immunogenic composition for use of according to any of the Embodiments E1-E8, wherein said self-assembling polypeptide derived from C4bp oligomerization domain comprises SEQ ID NO:2, or a functional variant thereof having at least 90% identity to SEQ ID NO:2.

E10. The immunogenic composition for use of according to any of the Embodiments E1-E9, wherein said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises the sequence of SEQ ID NO:4.

E11. The immunogenic composition for use according to any of the Embodiments E1-E10, wherein said carrier protein essentially consists of SEQ ID NO:5, or said carrier protein is a functional variant of SEQ ID NO: 5 having at least 90% identity to SEQ ID NO:5.

E12. The immunogenic composition for use according to any of the Embodiments E1-E11, wherein said fusion protein comprises or essentially consists of SEQ ID NO:6, or is a functional variant of SEQ ID NO: 6 having at least 90% identity to SEQ ID NO:6.

E13. The immunogenic composition for use according to any of the Embodiments E1-E12, wherein said amount of fusion protein is administered via intramuscular route.

E14. The immunogenic composition for use according to any of the Embodiments E1-E13, wherein said amount of fusion protein is administered as a single injection, preferably via intra-muscular route, to said human subject.

E15. The immunogenic composition for use according to any of the Embodiments E1-E14, wherein said subject is below 50 years old.

E16. The immunogenic composition for use according to any of the Embodiments E1-E15, wherein said subject is at least 50 years old, or above.

E17. The immunogenic composition for use according to any of the Embodiments E1-E16, wherein said use provides total T-cell response specific to NP, CD4 T-cell response specific to NP, anti-NP IgG (antibody response) and/or efficacy, protection or cross-protection from influenza symptoms (Influenza-Like Illness), in particular from influenza infection with influenza strain A or B.

E18. The immunogenic composition for use according to any of the Embodiments E1-E17, wherein said immunogenic composition is administered to a subject in combination concomitantly or sequentially, preferably concomitantly, with a second immunogenic composition against influenza comprising one or more inactivated strains of influenza, and/or an efficient amount of hemagglutinin HA antigen from one or more influenza strains, preferably said second immunogenic composition is Fluarix vaccine composition.

E19. An immunogenic composition comprising a fusion protein as defined in any one of Embodiments E1-E12, at a concentration of 300 μg/mL or above, and one or more pharmaceutically acceptable excipients.

E20. The immunogenic composition of Embodiment E19, wherein said fusion protein comprises at least 400 amino acid residues, for example between 400 and 600 amino acid residues, for example between 540 and 560 amino acid residues, optionally, said fusion protein forms protein nanoparticles with diameters comprised 20-100 nm and/or molecular weight of between 440-2200 kDa.

E21. The immunogenic composition of Embodiment E19 or E20, further comprising at least
i. a salt, e.g. sodium sulfate or sodium chloride, preferably sodium sulfate,
ii. an osmolyte, e.g. a sugar such as trehalose,
iii. a buffer, e.g. a phosphate buffer and/or a citrate buffer,
iv. optionally an antioxydant, e.g. methionine,
v. optionally a surfactant, e.g. polysorbate 80,
wherein the pH of the composition is between 6.0 and 7.0, typically between 6.3 and 6.6 and the osmolality is between 300 and 600 mOsm/kg, preferably between 400 and 500 mOsm/kg, for example about 450 mOsm/kg.

E22. The immunogenic composition of any one of Embodiments E19-E21, which comprises
i. sodium sulfate at a concentration of about 75 mM,
ii. trehalose at a concentration of about 200 mM,
iii. polysorbate 80 at a concentration between 0.02% and 0.08% (vol/vol), e.g. about 0.04%
iv. L-methionine at a concentration of about 5 mM.

E23. The immunogenic composition of any one of Embodiments E19-E22, wherein said composition does not comprise any adjuvant.

E24. The immunogenic composition of any one of Embodiments E19-E23, which is formulated as a ready-to-use sterile solution.

E25. The immunogenic composition of any one of Embodiments E19-E24, for use as a vaccine or immunotherapy in the prevention or treatment of influenza disease in a human subject in need thereof, in particular for use as defined in any one of Embodiments E1-E18.

### EXAMPLES

### Example 1: Development of a Stable Formulation of OVX836 at 300 μg/mL

OVX836 (SEQ ID NO:6) is the drug substance of a candidate vaccine comprising a fusion protein of OVX313 carrier protein (SEQ ID NO:5) and the seasonal flu Nucleoprotein (NP influenza virus A/Wilson-Smith/1933) fused to it.

OVX836 drug substance is supplied as a concentrated solution in a stabilizing formulation buffer. A first objective was to develop a stable formulation having a target concentration compatible with a single injection of up to 180 μg of OVX836 via intramuscular route.

Among the technical problems associated to the development formulation, one can mention the unusually high concentration that is targeted (300 μg/mL) and a quaternary configuration of OVX836 that is a dynamic equilibrium of heptamers (440 kDa) and small oligo-heptamers (di-, tri-, tetra-, or penta-heptamers), this feature being related to the self-associative properties of NP. Indeed, NP is a highly basic internal protein that provides structural and functional support to the viral replication machinery. To achieve this aim, NP forms homo-oligomers and multiple copies of NP wrap around genomic RNA. The quaternary structure of OVX836 thus results in a variety of morphologies, including small oligo-heptamers, that may cause polymerization into aggregates. There are several factors such as temperature, pH, ionic strength, concentration of protein which may affect this phenomenon of aggregation.

The first development formulation was prepared according to the recommended pH and osmolarity, respectively a pH close to 7.4 and osmolarity close to 300 mOsm.

As shown in the table 1 below, the formulation F1 was not stable and formed aggregates. Additional formulation with different buffers for more acidic pH were prepared with similar osmolarity (see Formulations F2, F3 and F4). However, all tested pH presented unsatisfactory stability and oligomerization.

TABLE 1
Quality attributes of OVX836 drug product within the first 3 months'
testing at the accelerated storage of 25° C.

Formulation

| Parameter | F1 | F2 | F3 | F4 |
|---|---|---|---|---|
| pH | 7.0 | 6.5 | 6.0 | 5.5 |

| | |
|---|---|
| Osmolality (mOsm/kg) | approx. 350 |

| | | | | |
|---|---|---|---|---|
| Protein content | ++ | +++ | +++ | +++ |
| Product degradation | + | ++ | ++ | ++ |
| Product oligomerization | + | ++ | + | + |

+++, stable;
++, moderately stable;
+, not very stable

We then opted for increasing the osmolality of the formulations. The results showed a significant improvement of both product degradation and product oligomerization at pH 6.5.

Formulation

| Parameter | F2-2 | F3-2 |
|---|---|---|
| pH | 6.5 | 6.0 |

| | |
|---|---|
| Osmolality (mOsm/kg) | approx. 450 |

| | | |
|---|---|---|
| Protein content | +++ | +++ |
| Product degradation | +++ | ++ |
| Product oligomerization | +++ | + |

+++, stable;
++, moderately stable;
+, not very stable

After screening different formulation buffers, excipients and pH, the following optimal formulation for stability was finally developed:

| Components | Concentration | | Function |
|---|---|---|---|
| OVX836 Drug Substance | 300 | μg/mL | Active Principle |
| Citric acid | 20 | mM | Buffer |
| Trisodium citrate | | | |
| Sodium dihydrogenophosphate | 7.5 | mM | Buffer |
| Dipotassium hydrogen phosphate | | | |
| Sodium sulfate | 75 | mM | Salt (ionic strength) |
| Trehalose | 200 | mM | Osmolyte |
| Polysorbate 80 | <0.08% (v/v) | | Surfactant |
| L-methionine | 5 | mM | Antioxydant |

| | | |
|---|---|---|
| Water for injection | — | Solvent |

More specifically, an optimal solubilization was achieved at a slightly acid pH between 5.5 and 7.0 (a pH between 6.4 and 6.6 being preferable). In addition, the screening showed that the use of a 20 mM Na citrate-based buffer (final pH value of 6.6) prevented the apparition of high-molecular weight oligomers in the medicinal product.

Moreover, in the development studies, the presence of trehalose was mainly found to slow down the oligomer formation of OVX836 (reducing the oligomerization as measured by size exclusion chromotagraphy analysis) and a concentration of 200 mM was found optimal.

Salts such as sodium chloride or sodium sulfate were also shown to be able to stabilize OVX836 but sodium sulfate was strongly preferred, as suggested by differential scanning calorimetry (DSC thermograms).

Stability data available of the selected optimal formulation showed no significant degradation of the OVX836 drug product, for at least 36 months when stored at 5° C. and 3 months at 25° C. The osmolality of the final formulation was between 440 and 500 mOsm/kg, typically between 465 and 480 Osm/kg.

A formulation with such high osmolality and high protein concentration may not be safe or well tolerated in human subjects. This was evaluated with the study detailed below in Example 2.

### Example 2: Phase 1-Randomized, Placebo-Controlled, Dose-Escalating Study to Evaluate OVX836, a Nucleoprotein-Based Influenza Vaccine: Intramuscular Results

| | OVX836-001 (completed) |
|---|---|
| Stage | Phase 1 (FIH) |
| Study design | Single center, randomized, observer blind, placebo-controlled |
| | study |
| Study IMP formulation | OVX836 300 μg/mL as disclosed in Example 1 |
| Doses, regimen & route | 30, 90 and 180 μg |
| | 2 injections at one month interval |
| | Intramuscual (IM)/Intranasal (IN) |
| | Placebo |
| No of subjects | 72 subjects (18-49 years old) |
| Primary objective | To evaluate the safety of 3 dose levels (30 μg, 90 μg, 180 μg) of |
| | OVX836 vaccine administered at Day 1 and Day 29 to healthy |
| | subjects either via IM or IN route. |
| Secondary objectives | To evaluate the immune response of 3 dose levels (30 μg, 90 μg, |
| | 180 μg) of OVX836 vaccine administered at Day 1 and Day 29 to |
| | healthy subjects either via IM or IN route. |
| Study status | Completed |

### Methods

This randomized, placebo-controlled, observer-blind, sequential, dose-escalation Phase 1 study was conducted at the University of Antwerp (Antwerpen, Belgium), in accordance with Good Clinical Practice. It was approved by the Ethics Committee of the Antwerp University Hospital and the University of Antwerp, and by the Belgian Federal Agency for Medicines and Health Products (FAMHP). An independent data and safety monitoring board regularly reviewed the data. Written informed consent was obtained from all participating subjects. The EudraCT number was 2018-000341-39 and the Clinicaltrials.gov number was NCT03594890.

Healthy adults aged 18-49 years, with a body mass index between 18 and 25 kg/m² were eligible for the study. The main exclusion criteria were previous influenza vaccination within 6 months before screening, pregnancy or unwillingness to practice birth control, positive test for the human immunodeficiency virus or hepatitis B/C viruses, presence of an acute febrile illness on the day of vaccination, treatments that could affect the immune response such as systemic corticosteroids, cytotoxic drugs, anti-inflammatory drugs and other immunomodulatory drugs, and history of significant medical illness such as autoimmune disorders, uncontrolled diabetes or hypertension, heart, renal, or hepatic diseases.

Twelve subjects were included into each of the three sequential cohorts (low dose 30 μg, medium dose 90 μg, high dose 180 μg). Each cohort was randomized at a 3:1 ratio between OVX836 vaccine (N=9) and placebo (N=3). The study was observer-blind. The investigator and the subject ignored the treatment arm (placebo/vaccine) the subject was allocated to, up to the end of the study (Month 5). Syringes containing the study product (placebo or vaccine) were prepared and administered by an unblinded team.

The vaccine (300 μg/mL active substance) or the placebo (consisting of sodium chloride 0.9%) was administered in the deltoid muscle of the non-dominant arm at low (30 μg in 0.1 mL), medium (90 μg in 0.3 mL) or high (180 μg in 0.6 mL) dose. The study was divided in two phases, an active treatment phase from Day 1 to Day 57, consisting of two intramuscular vaccinations, each followed by 28 days of follow-up, and a follow-up phase from Day 58 to Day 150 (Month 5) after 1st administration.

A diary card was used to collect solicited local (administration site pain, redness, swelling and induration) and systemic (fever, cough, headache, arthralgia, myalgia, malaise/tiredness and vomiting) symptoms that occurred within 7 days following each administration. Unsolicited adverse events (AEs) were recorded using open questions for 28 days after each administration. Intensities of AEs were graded as mild, moderate, severe or potentially life-threatening, and monitored throughout the active phase. Serious AEs (SAEs) were monitored throughout the study up to Month 5. A predefined set of safety laboratory analyses (hematology and clinical chemistry including coagulation parameters and evaluation of C-reactive protein (CRP)) was performed at screening and then on Days 8, 29, 36 and 57.

Whole blood samples were collected on Days 1, 8, 29, 36, 57 and 150 for isolation of PBMCs and determination of the NP-specific interferon-gamma (IFN-γ) T-cell response using an enzyme-linked immunospot assay (ELISPOT). Serum samples were collected on Days 1, 29, 57 and 150 for the determination of anti-NP, anti-OVX313 and anti-hC4BP IgG using enzyme-linked immunosorbent assays (ELISAs). Immunoassays are described in the Supplementary Methods.

The sample size was deemed sufficient given the purely exploratory nature of the study. The study was not powered for any statistical hypothesis testing. Descriptive statistics were used to summarize all relevant parameters: number and percentage for discrete variables and mean (arithmetic or geometric), median, standard deviation, 95% confidence interval (CI), minimum and maximum for continuous variables. Exploratory inferential analyses on immunogenicity data were performed using Kruskal-Wallis' tests at each time point to test overall difference between the treatment groups, followed when significant by post-hoc Dwass, Steel, Critchlow-Fligner's tests to test each pairwise comparison. Intragroup comparisons were performed using paired Wilcoxon's test. Fisher's exact tests were used to assess differences between the treatment groups in terms of percentage of responders. As no corrections were applied to take into account the multiplicity of endpoints and comparisons, p values<5% have to be considered only as indicative of potential statistically significant differences.

A total of 36 subjects were included and 33 subjects (91.7%) completed the whole study.

### Part 1: Preliminary Analysis

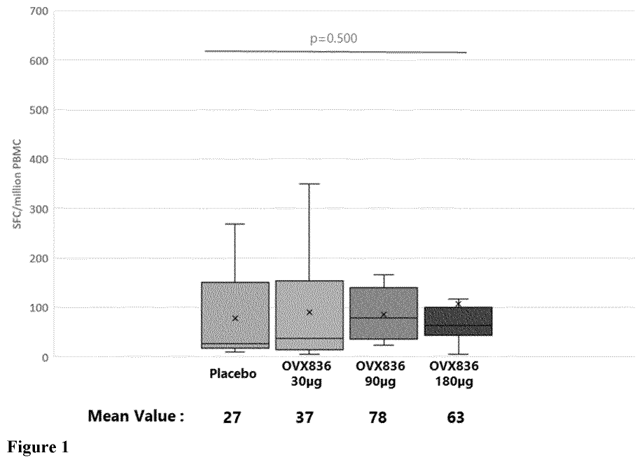

We conducted a first analysis of the results of Phase 1. All groups had similar baseline at day 1 before vaccination, which means that we could compare them (FIG. 1).

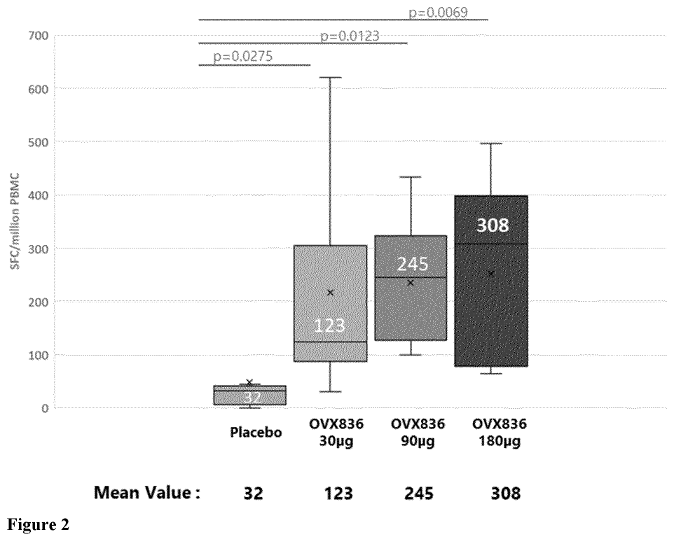

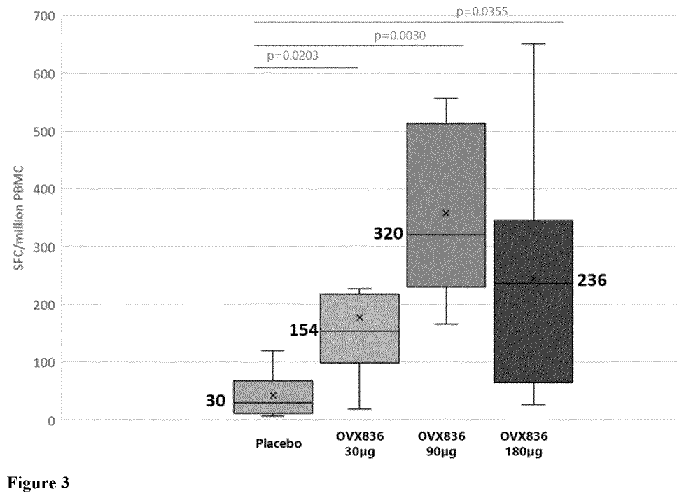

At day 8 and 36, all OVX836 groups showed a higher response than the placebo. At day 8, there was no difference between 90 and 180 μg groups but 30 μg seems a bit suboptimal (FIG. 2). At Day 36 (8 days after the second vaccination), there was no statistically significant differences between 90 and 180 μg and the 90 μg group even showed a slightly better response than the 180 μg group (FIG. 3).

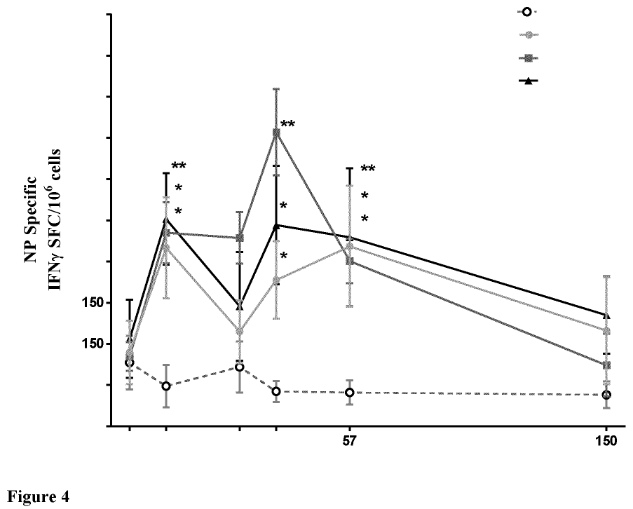

The FIG. 4 showed over-time evolution of the number of NP-specific IFN-γ Spot Forming T-cells (SFC)/10⁶ cells in the pooled placebo and the three OVX836 vaccinated groups (30 μg, 90 μg and 180 μg) from baseline (Day 1, pre-vaccination) to Day 150 (4 months after 2^{nd} administration). The results suggest that 90 μg might even be doing better than 180 μg after one single injection as shown by the sustained response at Day 29 vs others.

In summary, the preliminary analysis of the Phase I study demonstrated that:
A higher dose than 30 μg is required;
There was no significant advantage of a second injection;
Both 90 and 180 μg doses were safe and well tolerated with no dose effects;
There was no clear advantage of 180 μg dose as compared to 90 μg dose suggesting that a plateau for efficacy may have already been reached at the 90 μg dose;
The second dose of 90 μg triggered what may be a higher T-cell immune response than the 180 μg, suggesting that 90 μg may be better than the 180 μg dose level.

### Part 2: Detailed Analysis of the Phase I Results

Further analysis on the Phase 1 results has demonstrated the safety of the formulation and dosing regimen with 90 and 180 μg and surprisingly inferred a trend of dose response effect on immune response from 90 to 180 μg as detailed hereafter.

### Reactogenicity and Safety

No solicited local symptoms were reported in the placebo subjects, whereas most subjects vaccinated with OVX836 presented transient mild to moderate pain at the injection site. There was neither clear OVX836 dose-effect relationship in the number of solicited local symptoms nor in the number of affected subjects: 13 symptoms in 8 subjects at 30 μg, 22 symptoms in 7 subjects at 90 μg and 16 symptoms in 7 subjects at 180 μg. There was also no apparent increase in solicited local symptoms after the 2nd vaccination compared to the 1st one. None of the solicited local symptoms was severe (grade 3) in cohorts 1 (30 μg) and 3 (180 μg). Two solicited local symptoms (induration and oedema) were severe in one subject (11.1%) in cohort 2 (90 μg). None of the solicited local symptoms was ongoing at the end of the observation period after either vaccination.

There was neither dose-effect relationship in the number of solicited systemic symptoms nor in the number of affected subjects: 12 symptoms in 4 subjects at 30 μg, 13 symptoms in 6 subjects at 90 μg and 15 symptoms in 6 subjects at 180 μg. In comparison, 12 solicited systemic symptoms were reported by 5 out of the 9 placebo subjects. Two severe solicited systemic symptoms were reported in two subjects vaccinated with OVX836, each after the 1st vaccination: one severe malaise (tiredness) in cohort 1 (30 μg), and one severe fever (≥39° C.) in cohort 3 (180 μg); the latter led to suspension of the 2nd administration. None of the solicited systemic symptoms was ongoing at the end of the observation period after either vaccination.

The percentages of subjects reporting unsolicited AEs during the 28-day period after each vaccination was reported: No clear OVX836 dose-effect relationship could be observed. There was also no increase in unsolicited AEs after the 2nd versus the 1st vaccination.

Overall, 23 unsolicited AEs were reported in 8 subjects in cohort 1 (30 μg), 21 AEs in 8 subjects in cohort 2 (90 μg) and 21 AEs in 8 subjects in cohort 3 (180 μg), versus 25 AEs in 6 subjects in the pooled placebo groups. The following AEs were considered related to the vaccine: (i) Cohort 1—30 μg: injection site hemorrhage, vaccination site rash, musculoskeletal stiffness, CRP increase; (ii) Cohort 2—90 μg: injection site hemorrhage, nausea oropharyngeal pain, two cases of pre-syncope, CRP increase, neutrophil count decreased (severe), white blood cell (WBC) count decrease, and (iii) Cohort 3—180 μg: nasopharyngitis (severe), musculoskeletal pain, neck pain, pre-syncope, oropharyngeal pain, two events of nasal congestion, CRP increase, lymphocyte count decrease, neutrophil count increase, WBC count increase.

One SAE was reported in one OVX836 90 μg recipient consisting of a urinary tract infection occurring approximately 40 days after the 2nd vaccination. The SAE lasted for 11 days and was considered unrelated to the vaccine.

In conclusion, OVX836 appeared as a safe and well-tolerated candidate vaccine by the intramuscular route of administration, in a 30 μg to 180 μg dose range. No clear dose-effect relationship has been demonstrated and the 180 μg dose did not appear to be the maximum tolerated dose.

### NP-Specific T-Cell Immune Response

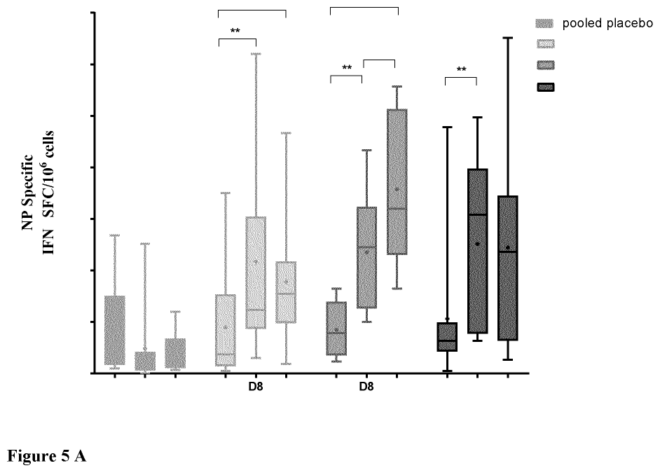

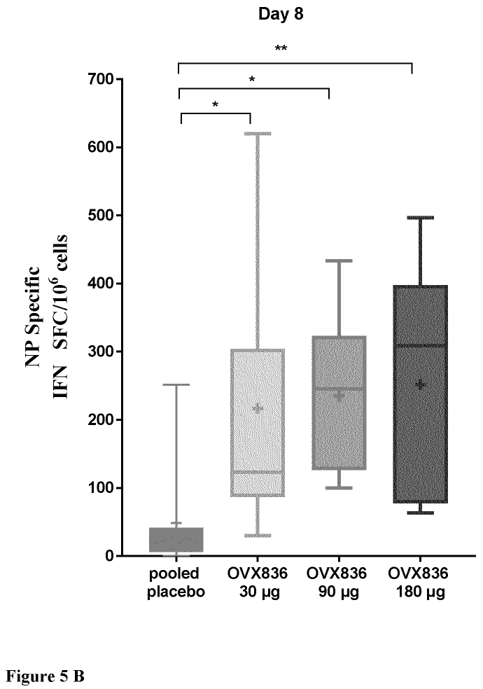

The number of NP-specific IFN-γ-producing T-cells detected on Day 1, Day 8 (1-week after the 1st vaccination) and Day 36 (1-week after the 2nd vaccination) are shown for the three OVX836 vaccinated groups versus placebo in FIG. 5. All subjects had pre-existing NP-specific IFN-γ producing T-cells at baseline, ranging from 5 to 478 NP-specific IFN-γ spot-forming cells (SFCs)/10⁶ PBMCs, with no significant difference between groups. On Day 8 after the 1st vaccination, there was a significant increase in the mean SFCs/10⁶ PBMCs in each of the three OVX836 vaccine groups versus Day 1 (FIG. 5A) and compared to placebo (FIG. 5B). There was a trend for an increase of the response as a function of the OVX836 dose-level at Day 8, but this effect was not significant. The 2nd vaccination did not allow to further increase the response on Day 36 (1 week after 2nd vaccination), except for the OVX836 90 μg group. On Day 57 (28 days after 2nd vaccination), significant differences were found in the three vaccine groups versus placebo (p=0.002 overall; Kruskal-Wallis test), with no significant differences between OVX836 groups. On Day 150 (4 months after 2nd vaccination), the number of NP-specific IFN-γ-producing T-cells were still above the placebo in the 3 OVX836 groups, but the difference was not more statistically significant (p=0.295 overall; Kruskal-Wallis test).

### NP-Specific Humoral Immune Response

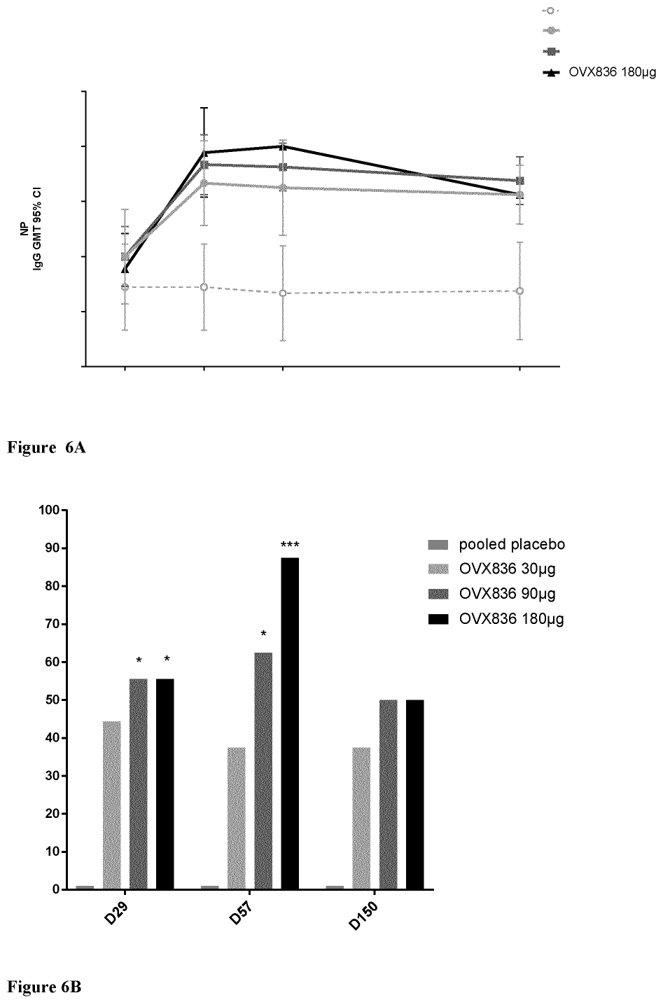

The over-time evolution of anti-NP IgG geometric mean titers (GMTs) in the three OVX836 vaccine and placebo groups is shown in FIG. 6 (Panel A). All subjects presented pre-existing anti-NP IgG at baseline, individual titers ranging from 1,600 to 25,600, with no significant difference between groups. On Day 29 after the 1st vaccination, there was a significant increase in GMTs in the three vaccine groups compared to placebo (p=0.0008 overall; Kruskal-Wallis test). The 2nd vaccination on Day 29 did not allow to further increase the anti-NP IgG GMTs on Day 57 (28 days after 2nd vaccination), which remained high at Day 150 (4 months after 2nd vaccination), still significantly higher in the three vaccine groups as compared to placebo (p=0.001 overall; Kruskal-Wallis test). There was a trend for an increase of the anti-NP IgG GMTs as a function of the OVX836 dose-level, but this effect was not significant.

The percentage of subjects with a four-fold increase in anti-NP IgG titers at the different time points post-vaccination versus baseline is shown in FIG. 6 (Panel B). On Day 29 (after 1st vaccination) and Day 57 (28 days after 2nd vaccination), between 44.4% and 87.5% of OVX836 vaccinated subjects presented a 4-fold increase of their baseline titer, versus 0% in the placebo group. The overall difference between groups was significant at these two time points (p=0.035 at Day 29 and p=0.001 at Day 57; Kruskal-Wallis test), post-hoc statistical tests showing significant differences between OVX836 90 μg and OVX836 180 μg versus placebo. On Day 150 (4 months after 2nd vaccination), between 37.5 and 50.0% of OVX836 vaccinated subjects still presented a 4-fold increase of their baseline titer, versus 0% in the placebo group, but the difference between the 4 groups was not more statistically significant (p=0.128; Kruskal-Wallis test).

In summary, the detailed analysis of the results of the Phase I study demonstrated that:
Intramuscular route is preferred as intranasal route;
A higher dose than 30 μg is required;
Both 90 and 180 μg doses were safe and well tolerated with no dose effects;
There was no significant advantage of a second injection;
There was a trend of dose response between 90 and 180 μg.

### Example 3: Phase 2a Study

### A. Summary of the Study

| | OVX836-002 (completed) |
|---|---|
| Stage | Phase 2a |
| Study design | Single center, randomized, double blind, active controlled study |
| Study IMP formulation | OVX836 300 μg/mL |
| Doses, regimen & route | 90 & 180 μg |
| | Single injection |
| | IM |
| | Active: Influvac Tetra ™ |
| No of subjects | 300 subjects (18-65 years old) |
| Primary objective | Evaluate the immunogenicity of one administration of OVX836 |
| | vaccine at two dose levels (90 μg and 180 μg), in comparison to |
| | Influvac Tetra ™, 7 days after IM administration. |
| Secondary objectives | To evaluate the safety and reactogenicity of all |
| | investigational vaccines in the study. |
| | To evaluate NP immune response at the two dose levels |
| | (90 μg and 180 μg) of OVX836. |
| | To evaluate the immune response to all the investigational |
| | vaccines in the study in two age strata: subjects aged 18 to |
| | 49 years and subjects aged 50 to 65 years. |
| Study status | Clinical Study Report ongoing |

### B. Summary of the Results

In contrast to the preliminary analysis of the Phase I results, the Phase 2a has clearly demonstrated a significant dose response effect on immune response from 90 to 180 μg.

In particular, a strong increase of NP-specific T-cell responses and more particularly NP-specific CD4+ T cell responses were observed 8 days after injection, with a dose response between 90 and 180 μg.

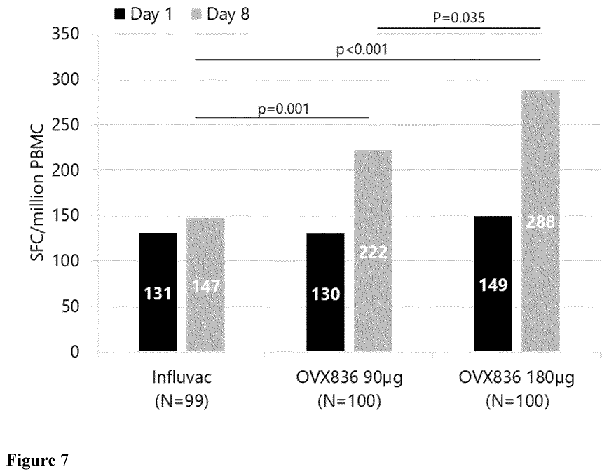

In the ITT cohort (Intent-to-Treat Cohort) after elimination of two outlier subjects in the OVX836 180 μg group (subjects 128-095 and 232-365 presenting high baseline values on Day 1:957 and 1630, respectively), in terms of response kinetics (pooled age strata) in the OVX836 90 μg group, the mean increased from 130 SFC/million PBMC at baseline to 222 SFC/million PBMC at Day 8. In the OVX836 180 μg group, the mean increased from 149 at baseline to 288 SFC/million PBMC at Day 8. In the Influvac Tetra group, the mean remained relatively stable at 131 SFC/million PBMC at baseline, and 147 SFC/million PBMC at Day 8. On Days 8, OVX836 180 μg was significantly different from OVX836 90 μg (p=0.035), supporting a dose-response relationship in the OVX836 groups in all subjects. The FIG. 7 shows the results at day 1 and day 8.

In the Per Protocol-D29 (PP-D29) cohort, in terms of response kinetics (pooled age strata) in the OVX836 90 μg group, the median (mean±SD) increased from 90 (131±153) SFC/million PBMC at baseline to 167 (223±191) and 163 (208±183) SFC/million PBMC at Days 8 and 29, respectively. In the OVX836 180 μg group, the median (mean±SD) increased from 95 (168±242) SFC/million PBMC at baseline to 200 (294±275) and 190 (278±245) SFC/million PBMC at Days 8 and 29, respectively. Then, in both OVX836 groups, the response waned down to the baseline on Day 180. In the Influvac Tetra group, the median (mean±SD) remained relatively stable at 96 (137±153) SFC/million PBMC at baseline, and 108 (147±149), 94 (162±206) and 81 (121±131) SFC/million PBMC at Days 8, 29 and 180, respectively. Subjects aged 18-49 years, PP-D29 cohort: In the OVX836 90 μg group, the median (mean±SD) increased from 102 (138±148) SFC/million PBMC at baseline to 198 (252±208) and 175 (205±169) SFC/million PBMC at Days 8 and 29, respectively. In the OVX836 180 μg group, the median (mean±SD) increased from 97 (152±174) SFC/million PBMC at baseline to 198 (275±239) and 202 (260±210) SFC/million PBMC at Days 8 and 29, respectively. In the Influvac Tetra group, the median (mean±SD) remained relatively stable at 102 (138±155) SFC/million PBMC at baseline, and 108 (137±134) and 106 (169±221) SFC/million PBMC at Days 8 and 29, respectively. From a statistical perspective, the effect of time (p<0.0001), treatment (p=0.0471) and time-treatment interaction (p<0.0001) was significant. There were no significant differences between group means on Day 1 (all p>0.05). On Day 8, the differences between OVX836 90 μg and Influvac Tetra (p=0.0017) and between OVX836 180 μg and Influvac Tetra (p=0.0001) were significant. On Day 29, only the difference between OVX836 180 μg and Influvac Tetra was significant (p=0.0202).

Subjects aged 50-65 years, PP-D29 cohort: In the OVX836 90 μg group, the median (mean±SD) increased from 62 (114±168) SFC/million PBMC at baseline to 107 (152±113) and 133 (216±219) SFC/million PBMC at Days 8 and 29, respectively. In the OVX836 180 μg group, the median (mean±SD) increased from 93 (209±370) SFC/million PBMC at baseline to 222 (345±354) and 171 (328±321) SFC/million PBMC at Days 8 and 29, respectively. In the Influvac Tetra group, the median (mean±SD) remained relatively stable at 84 (137±151) SFC/million PBMC at baseline, and 103 (172±186) and 79 (144±159) SFC/million PBMC at Days 8 and 29 respectively. From a statistical perspective, the effect of time (p=0.0004) and time-treatment interaction (p=0.0479) was significant. The effect of treatment was not significant (p=0.0964). There were no significant differences between group means on Day 1 (all p>0.05). On Day 8, only the difference between both OVX836 dose levels was significant (p=0.0217). On Day 29, only the difference between OVX836 180 μg and Influvac Tetra was significant (p=0.0372).

Although non-significant from a statistical perspective, there was a trend for a dose-effect relationship in the OVX836 groups in all subjects (observed in both age strata as well).

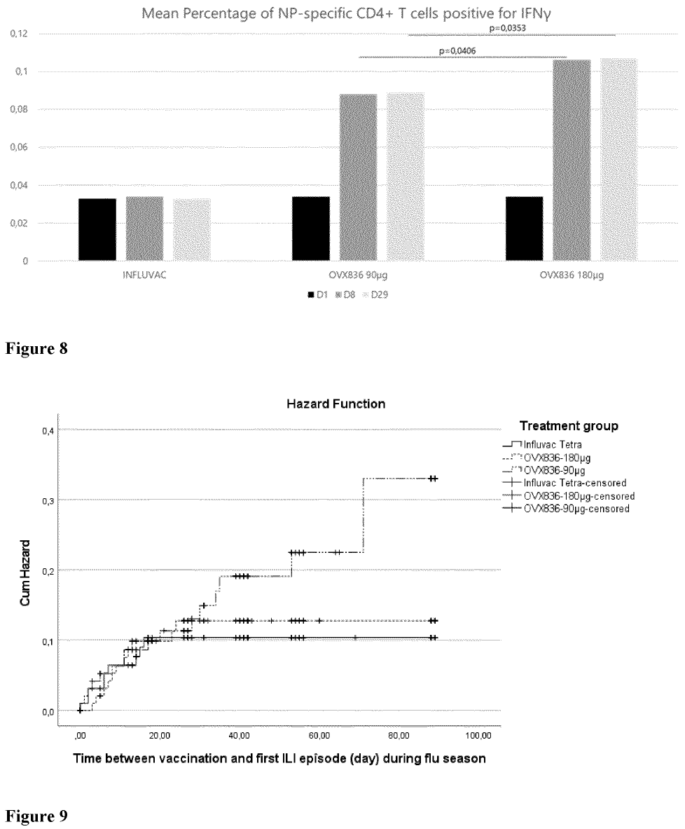

In the Per Protocol-D29 (PP-D29) cohort, baseline (pre-vaccination) percentages of NP-specific CD4+ T-cells expressing IFNγ were low and very similar between treatment groups. There was no effect at all of the vaccine in the Influvac Tetra group. In the OVX836 90 μg group, median (mean±SD) was increased from 0.022% (0.034±0.043%) at baseline to 0.075% (0.088±0.063%) on Day 8 and to 0.075% (0.089±0.057%) on Day 29. In the OVX836 180 μg group, there was an increase from 0.028% (0.034±0.027%) at baseline to 0.083% (0.106±0.076%) on Day 8 and to 0.096% (0.107±0.070%) on Day 29. Then, in both OVX836 groups, the response waned down to a value still slightly higher than the baseline on Day 180 (0.040% [0.048%±0.028%] in the OVX836 90 μg group and 0.050% [0.055%±0.032%] in the OVX836 180 μg group). As shown in FIG. 8, on Days 8 and 29, OVX836 180 μg was significantly different from OVX836 90 μg (p=0.0406 and p=0.0353, respectively), supporting the dose-response relationship already mentioned above for the total T-cells response (observed in age strata below and over 50 years old as well). Such NP-specific polyfunctional CD4 T-cell responses sustained 6 months after vaccination. The results also showed a strong and long-term increase of anti-NP IgG for all doses of OVX836.

### Safety Results

In addition, the Phase 2a confirmed an absence of dose-response effect on safety, indicating an excellent safety profile, similar to licensed seasonal flu vaccine for both tested doses of 90 and 180 μg.

### Efficacy Results

Most interestingly, a threshold effect was shown for a protective efficacy of OXV836 at 180 μg against influenza-like illness (ILI) symptoms.

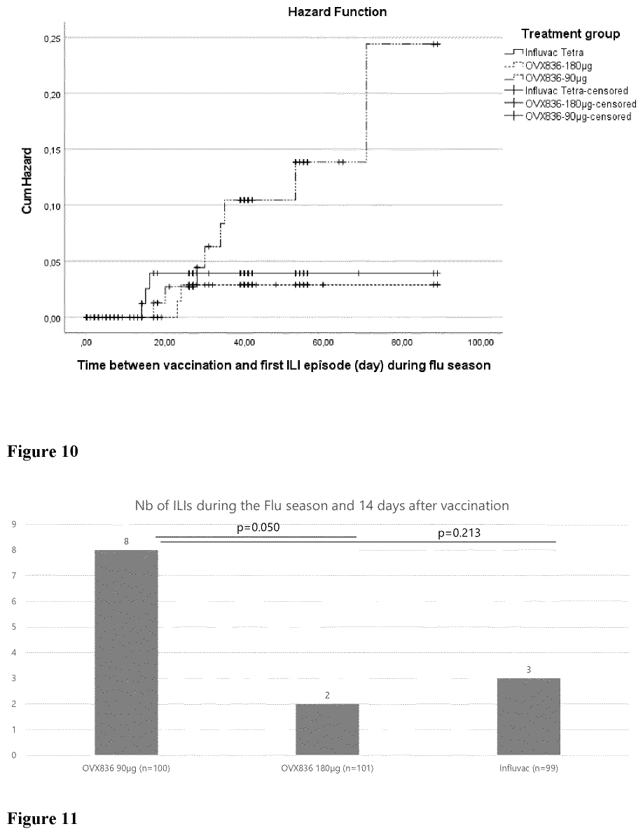

More specifically, Kaplan-Meier survival analyses were used to evaluate the cumulative hazard of non-specific ILIs as a function of time during the influenza season. Two analyses were performed. The first took into account all ILIs occurring during the 2019-2020 flu season up to 9 Mar. 2020 (FIG. 9) and the second took into account the ILIs occurring during the same period but from 14 days post-vaccination (FIG. 10). It is indeed commonly admitted that vaccines start protecting the subjects roughly two weeks after vaccination. Log-rank tests were used to compare the three treatment groups. When all ILIs occurring during the flu season were considered, all comparisons between treatment groups were non-significant (p=0.325). When ILIs occurring during the same period but from 14 days post-vaccination were taken into account, there was a trend for a difference between the three groups, although non-statistically significant (p=0.088), in particular the difference between OVX836 90 μg and OVX836 180 μg (p=0.054) and the difference between OVX836 90 μg and Influvac Tetra (p=0.130), while the OVX836 180 μg and Influvac Tetra had very similar profiles (p=0.650).

In terms of number of ILIs during the Flu season and more than 14 days after vaccination, the OVX836 90 μg group reached higher values compared to the OVX836 180 μg and Influvac Tetra group, which had a similar profile (8, 2 and 3 ILIs during the influenza season and from 14 days post-vaccination respectively in the OVX836 90 μg, OVX836 180 μg and Influvac Tetra groups, see FIG. 11). This could reveal a potential signal of efficacy of OVX836 at the dose of 180 μg. This need of course to be explored in further clinical trials.

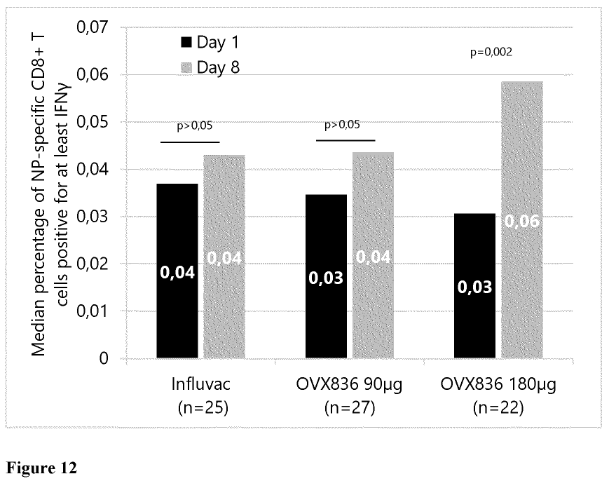

Lastly, in sub-population analysis, FIG. 12 shows that in subjects belonging to the lowest quartile of the CD8+ response at baseline (most likely the ones with the lowest probability of recent exposure/infection by influenza virus preceding vaccination), the median percentage of NP-specific CD8+ T-cells expressing IFNγ increased significantly (p=0.020) in the OVX836 180 μg group only

In Summary, the Results of Phase 2a Highlights a Strong Rationale to Test a Dose Version of OVX836 Higher than 180 μg. This Rationale can be Summarized as Follow:

### 1. Immunogenicity: Strong Increase in NP-Specific Immune Response

Dose-response effects with 180 μg superior to 90 μg for NP-specific Total T-cell (ELISpot), CD4 T-cell (ICS) and IgG (trend for the latter)
Dose-response effects with 180 μg significant in the lowest quartile of the CD8+ response at baseline (subpopulation analysis of FIG. 12)
   2. Efficacy: Decrease of ILIs During the Flu Season with OVX836 180 μg Superior to 90 μg
Trend of difference between 180 and 90 μg for the cumulative risk of ILIs
Significant difference in terms of number of ILIs during the flu season between 180 and 90 μg

### 3. Safety: OVX836 is Well Tolerated in all Patients and at all Doses (Up to 180 μg)

No vaccine related serious adverse events
Comparable to commercial vaccine
No evidence for disease enhancement detected
   Altogether, these Data Suggested to Investigate Higher Doses of OVX836 as a New Development Program as Described in Example 4

### Example 4: Second Phase I/Phase 2a Study (OVX836-003)

| Stage | Phase 2a |
|---|---|
| Study design | Single center, open-label, parallel groups study controlled versus |
| | regimen evaluated in OVX836-001 & 002 (180 μg IM), controlled |
| | versus placebo |
| Study IMP formulation | OVX836 |
| Doses, regimen & route | 180 μg & 300 μg & 480 μg |
| | Single injection |
| | IM |
| | Placebo |
| No of subjects | 136 (18-55 years old) |
| | 100 (65 years old and above) |
| Primary objective | Evaluate the immunogenicity of one administration of OVX836 |
| | influenza vaccine at two dose levels (300 μg and 480 μg) given IM, |
| | in comparison to OVX836 influenza vaccine at 180 μg given IM and |
| | placebo. |
| Secondary objectives | To evaluate the safety and reactogenicity of the |
| | investigational vaccine at three dose levels (180 μg, 300 μg |
| | and 480 μg) in the study, in comparison to placebo. |
| | To evaluate NP immune responses of one single |
| | administration of OVX836 vaccine at two dose levels |
| | (300 μg and 480 μg) given IM each in comparison to |
| | OVX836 influenza vaccine at 180 μg given IM and to |
| | placebo. |
| Primary Outcome | 1. Change of NP-specific T-cell frequencies in peripheral blood, |
| measure | measured by IFNγ ELISPOT, at Day 8 versus pre-injection |
| | baseline (Day 1). |
| | [Time Frame: at Day 8 versus pre-injection baseline (Day 1)] |
| | 2. Proportion of subjects reporting solicited local (Injection site |
| | redness, Injection site swelling, Injection site pain) and systemic |
| | symptoms (Fatigue, Headache, Arthralgia, Malaise, Myalgia, |
| | Fever) |
| | [Time Frame: during 7 days after vaccine administration] |
| | 3. Proportion of subjects reporting unsolicited Adverse Events |
| | [Time Frame: during 29 days after vaccine administration] |
| | 4. Proportion of subjects with Influenza-Like-Illness cases |
| | associated with laboratory-confirmed influenza |
| | [Time Frame: during the whole study duration, 180 days] |
| | 5. Severity scores of Influenza-Like-Illness cases (as per Flu-PRO ® |
| | questionnaire) |
| | [Time Frame: during the whole study duration, 180 days] |
| | 6. Proportion of subjects reporting Serious Adverse Events |
| | [Time Frame: during the whole study duration, 180 days] |
| Secondary outcome | 7. Change of NP-specific T-cell frequencies in PBMCs, measured |
| measure | by IFNγ ELISPOT, at Day 8 versus pre-injection baseline (Day 1). |
| | [Time Frame: at Day 8 versus pre-injection baseline (Day 1)] |
| | 8. NP-specific T-cell frequencies (measured by IFNγ ELISPOT on |
| | PBMCs) at Day 1 (pre-injection baseline), Day 8 and Day 180 |
| | [Time Frame: at Day 1 (pre-injection baseline), Day 8 and Day 180] |
| | 9. NP-specific CD4+ and CD8+ T-cell frequencies measured by flow |
| | cytometry (on PBMCs) as expressing IL-2, TNF & and/or IFNγ |
| | upon in vitro stimulation at Day 1 (pre-injection baseline), Day 8 and |
| | Day 180. |
| | [Time Frame: at Day 1 (pre-injection baseline), Day 8 and Day 180] |
| | 10. Geometric mean titers (GMTs) of anti-NP Immunoglobulin G |
| | (IgG) (ELISA, serum) at Day 1 (pre-injection baseline), Day 8, Day |
| | 29 and Day 180 |
| | [Time Frame: at Day 1 (pre-injection baseline), Day 8, Day 29 and |
| | Day 180] |
| | 11. Proportion of subjects with an increase (two-fold and four-fold) |
| | in anti-NP Immunoglobulin G (IgG) (ELISA, serum) titer with |
| | respect to pre-injection baseline (Day 1), at Day 8, Day 29 and Day |
| | 180 |
| | [Time Frame: at Day 8, Day 29 and Day 180 versus pre-injection |
| | baseline (Day 1)] |
| Study status | Ongoing |

### Results

Safety: All dosages (180, 300 and 480 μg) of OVX836 were found safe, well-tolerated and comparable to seasonal quadrivalent influenza vaccine Influvac Tetra™. Low incidence of “severe” (Grade 3 as per FDA toxicity scale for vaccine clinical trials) adverse events and no dose-limiting effects.

Immunogenecity: In contrast to the preliminary analysis of the Phase I results, this second Phase 2a has clearly demonstrated a significant dose response effect on immune response beyond the dose of 180 μg and up to 480 μg.

In particular, a strong increase of NP-specific T-cell responses and more particularly NP-specific CD4+ T cell responses were observed 8 days after injection, with a dose response between 180 and 480 μg. Besides, NP-specific CD8+ T-cell responses (IFNγ+/IL2+/TNFα-CD8 T-cells) were observed 8 days after injection for the 300 μg and 480 μg dose-level, which differs from the observation with the 180 μg dose-level for which no response was observed 8 days after injection.

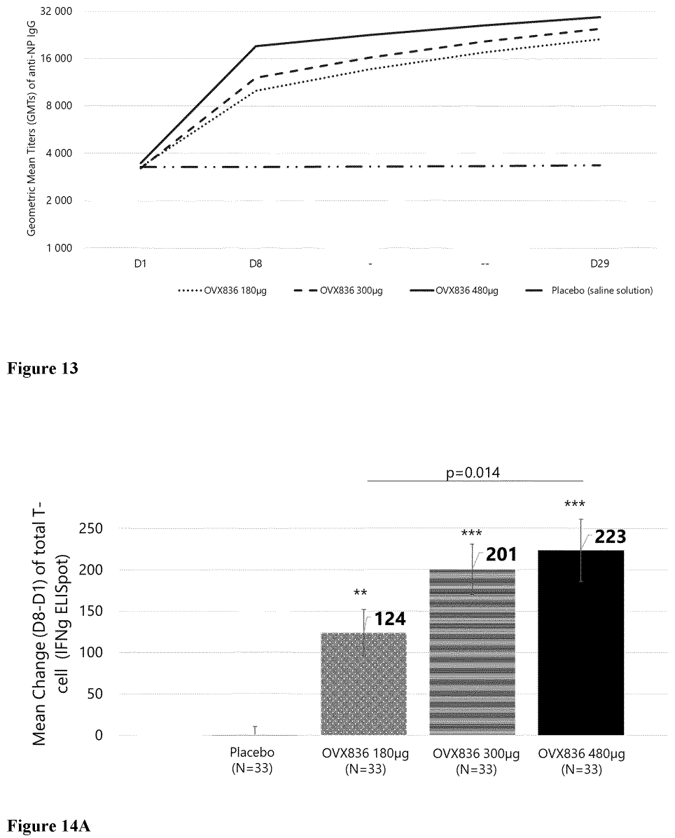

Using per protocol Bonferroni's intergroup pairwise comparison statistics, on Days 8, OVX836 480 μg was significantly different from OVX836 180 μg (p=0.026) in terms of anti-NP IgG response, supporting the dose-response relationship beyond 180 μg dose-level (FIG. 13). Although non-significant from a statistical perspective, there was a trend for a dose-effect relationship between the OVX836 480 μg and 180 μg groups for the ratio of % of positive CD4 T-cells at Day 8 vs Day 1, specifically for IFNγ+/IL2+ TNFα-CD4 T-cells (p=0.083) and for polypositive CD4 T-cells (p=0.102).

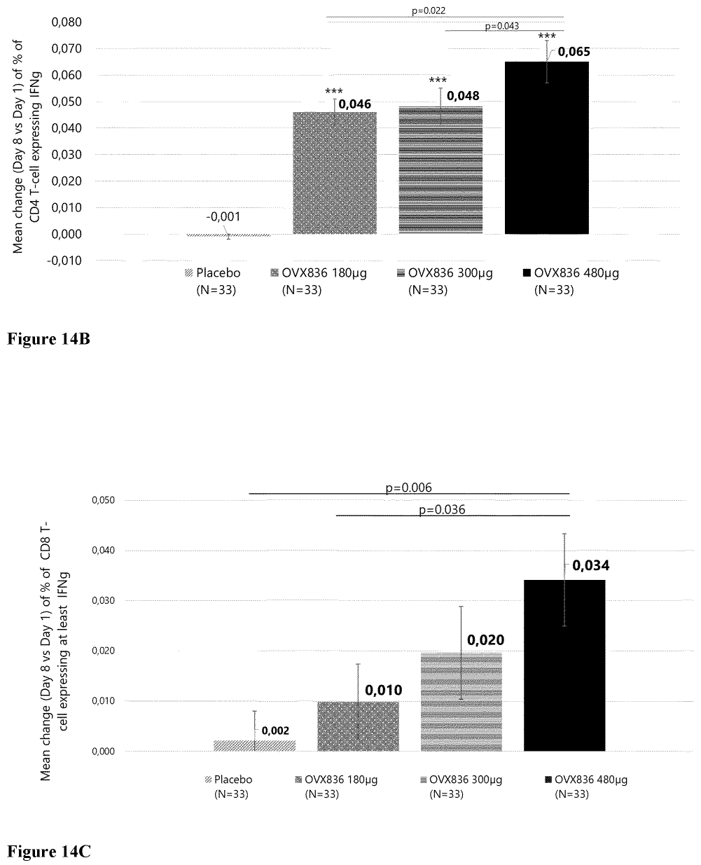

Furthermore, a dose-effect relationship was observed between 180 μg and higher dose-levels of OVX836 in terms of change between Day 1 and Day 8 of T-cell responses (total T-cell, CD4 and CD8 T-cells) when applying exploratory inferential analyses on immunogenicity data using ANOVA test between groups to test difference between the treatment groups and between timepoints, followed when significant (p<0.05) by pairwise Fisher's LSD comparison to assess differences between the treatment groups. As no corrections were applied to take into account the multiplicity of endpoints and comparisons, p values<5% have to be considered only as indicative of potential statistically significant differences:
ELISpot IFNγ responses (FIG. 14A): there was no effect at all of the placebo. In the OVX836 180 μg group, mean change between Day 1 and Day 8 was 124 SFC per million PBMC (p=0.002 vs Placebo) while the increase was respectively 201 and 223 SFC per million PBMC for the 300 μg and 480 μg dose-levels (p<0.001 vs Placebo for both dose-levels). A significant difference was observed between the 480 μg and 180 μg (p=0.014)
CD4 T-cell responses (FIG. 14B): there was no effect at all of the placebo. In the OVX836 180 μg group, mean change of % of CD4 T-cell positive for IFNγ between Day 1 and Day 8 was 0.046 (p<0.001 vs Placebo) while the increase was respectively 0.048 and 0.065 for the 300 μg and 480 μg dose-levels (p<0.001 vs Placebo for both dose-levels). A significant difference was observed between the 480 μg and 180 μg (p=0.022) and between 480 μg and 300 μg (p=0.043)
CD8 T-cell responses (FIG. 14C): there was no effect of the placebo nor of the 180 μg or 300 μg dose-levels. In the OVX836 480 μg group, mean change of % of CD8 T-cell positive for both IFNγ and IL2 between Day 1 and Day 8 was 0.034 (p=0.006 vs Placebo). A significant difference was observed between the 480 μg and 180 μg (p=0.036) in terms of mean change of % of CD8 T-cell positive for both IFNγ and IL2 between Day 1 and Day 8.

Efficacy: An observational study (FLU-001 study) was run in parallel with the OVX836-003 study (same site, same timing of recruitment, same inclusion/exclusion criteria), with the objective to merge the two cohorts in the event where influenza was actively circulating in order to make an analysis on ILIs on an equilibrated set of 200 subjects (50% OVX836 at doses higher than 180 μg; 50% of placebo or untreated subjects).

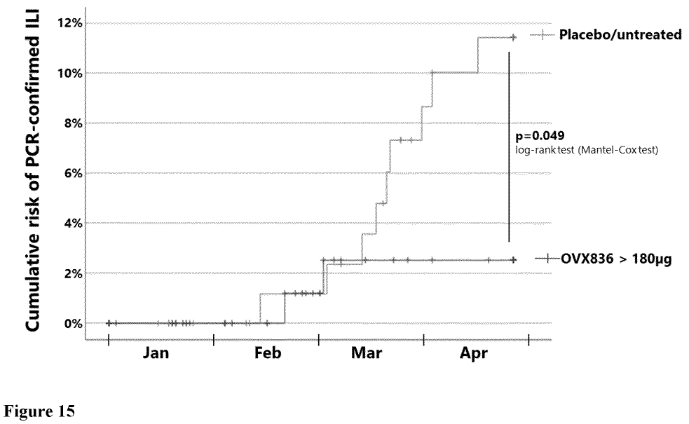

Two cases of PCR-confirmed symptomatic Influenza (ILIs) were reported in the OVX836 groups (all dose-levels) vs 9 cases for the placebo+untreated cohorts, reflecting an Observed Efficacy of 79% [5.4%; 95.4%] (see FIG. 15).

### Example 5: Phase 2a Study (OVX836-004)

| Stage | Phase 2a |
|---|---|
| Study design | Phase 2a, Randomized, Double-blind (Double-dummy), Controlled, |
| | Parallel-group Study to Evaluate the Immunogenicity and the |
| | Safety of the Concomitant Administration of OVX836 Influenza |
| | Vaccine and a Quadrivalent Inactivated Influenza Vaccine Given |
| | Intramuscularly as 2 Separate Injections in the Same Arm, in |
| | Comparison to Co-administration of Quadrivalent Inactivated |
| | Influenza Vaccine and Placebo and to Co-administration of |
| | OVX836 and Placebo Given Intramuscularly in Healthy Subjects. |
| Study IMP formulation | OVX836 |
| Doses, regimen & route | Biological/Vaccine: OVX836 480 μg |
| | One single administration intramuscularly at Day 1 |
| | Biological/Vaccine: Quadrivalent Inactivated Influenza Vaccine |
| | (Fluarix ® Tetra) |
| | One single administration intramuscularly at Day 1 |
| | Vs Active Comparator |
| | Biological/Vaccine: Quadrivalent Inactivated Influenza Vaccine |
| | (Fluarix ® Tetra) |
| | One single administration intramuscularly at Day 1 |
| | Biological/Vaccine: Placebo |
| | One single administration intramuscularly at Day 1 |
| | And Placebo Comparator |
| | Biological/Vaccine: OVX836 480 μg |
| | One single administration intramuscularly at Day 1 |
| | Biological/Vaccine: Placebo |
| | One single administration intramuscularly at Day 1 |
| No of subjects | 180 (anticipated) |
| Primary Outcome | Number of seroconversion determined using Hemagglutination- |
| Measure | Inhibition assay, for the four influenza strains contained in the |
| | Quadrivalent Influenza Vaccine. |
| | Seroconversion is defined as a negative pre-vaccination |
| | Hemagglutination-Inhibition assay titer and post-vaccination |
| | Hemagglutination-Inhibition assay titer ≥1:40, or a fourfold increase |
| | in Hemagglutination-Inhibition assay titer between pre- and post- |
| | vaccination timepoints. |
| | [Time Frame: At Day 29 versus pre-injection baseline (Day 1)] |
| | 2. Proportion of subjects achieving a titer ≥1:40 at Day 29 |
| | determined using Hemagglutination-Inhibition assay, for the four |
| | influenza strains contained in the Quadrivalent Influenza Vaccine. |
| | [Time Frame: At Day 29] |
| | 3. Number of Hemagglutination-Inhibition assay titers geometric |
| | mean ratios >2.5 for the four influenza strains contained in the |
| | Quadrivalent Influenza Vaccine. |
| | [Time Frame: At Day 29 versus pre-injection baseline (Day 1)] |
| | 4. Proportion of subjects reporting solicited local (Injection site |
| | redness, Injection site swelling, Injection site pain) and systemic |
| | signs and symptoms (Fatigue, Headache, Arthralgia, Malaise, |
| | Myalgia, Fever) |
| | [Time Frame: During 7 days after vaccine administration] |
| | 5. Proportion of subjects reporting unsolicited AEs |
| | [Time Frame: During 29 days after vaccine administration] |
| | 6. Proportion of subjects with Influenza-Like-Illness cases |
| | [Time Frame: During the whole study duration, 180 days] |
| | 7. Severity scores of Influenza-Like-Illness cases (as per Flu-PRO |
| | questionnaire) |
| | [Time Frame: During the whole study duration, 180 days] |
| | 8. Proportion of subjects reporting Serious Adverse Events |
| | [Time Frame: During the whole study duration, 180 days] |
| Secondary Outcome | 9. Hemagglutination-Inhibition assay geometric mean titers for each |
| measure | of the four strains contained in the Quadrivalent Influenza Vaccine. |
| | [Time Frame: At Day 1 (pre-injection baseline) and Day 29] |
| | 10. Cell-mediated immune response in terms of change of |
| | Nucleoprotein-specific T-cell frequencies in Peripheral Blood |
| | Mononuclear Cells, measured by Interferon Gamma Enzyme- |
| | Linked Immunospot Assay. |
| | [Time Frame: At Day 8 versus pre-injection baseline (Day 1)] |
| | 11. Geometric Mean Titer of anti-Nucleoprotein immunoglobulin G |
| | (Enzyme-Linked Immunosorbent Assay, serum). |
| | [Time Frame: At Day 1, Day 8 and Day 29] |
| | 12. Proportion of subjects with an increase (four-fold) in anti- |
| | Nucleoprotein Immunoglobulin G (Enzyme-Linked Immunosorbent |
| | Assay, serum) titer. |
| | [Time Frame: At Day 29 with respect to pre-injection baseline (Day |
| | 1)] |
| Study status | Not yet started |

### Results

Safety: All arms (Quadrivalent Inactivated Influenza Vaccine (QIIV), OVX836 & QIIV, and OVX836) were found safe and well-tolerated with low incidence of “severe” (Grade 3 as per FDA toxicity scale for vaccine clinical trials) adverse events (one severe Fatigue/Myalgia in the QIIV arm, and one severe Headache in the OVX836 arm) while no “serious” (Grade 4 as per FDA toxicity scale for vaccine clinical trials) adverse events were reported in the study.

Efficacy: Three cases of PCR-confirmed symptomatic Influenza (ILIs) were reported in the QIIV arm vs 1 case for the OVX836 arm, and vs 2 cases for the OVX836 & QIIV arm.

### Example 6: Useful Sequences for Practicing the Invention

TABLE 2
Useful sequences for practicing the invention

| SEQ ID NO: | Type | Brief Description |
|---|---|---|
| 1 | aa | Amino acid sequence of the nucleoprotein antigen (sequence as used in |
| | | OVX836 without SP sequence) |
| 2 | aa | Amino acid sequence of the hybrid C4bp oligomerization domain without the |
| | | C-terminal positively charged tail (including Glutamate as last residue) |
| 3 | aa | Amino acid sequence of the C-terminal positively charged tail (ZXBBBBZ) |
| 4 | aa | Amino acid sequence of the C-terminal positively charged tail (GRRRRRS) |
| 5 | aa | OVX313 full amino acid sequence without GS linker |
| 6 | aa | OVX836 full amino acid sequence without methionine |
| 7 | nt | Nucleotide sequence encoding the nucleoprotein antigen (sequence as used |
| | | in OVX836 without methionine) |
| 8 | nt | Nucleotide sequence encoding the hybrid C4bp oligomerization domain |
| | | without the C-terminal positively charged tail (including Glutamate as last |
| | | residue) |
| 9 | nt | Nucleotide sequence encoding the C-terminal positively charged tail |
| | | (GRRRRRS) |
| 10 | nt | OVX313 full nucleotide coding sequence without GS linker |
| 11 | nt | OVX836 full nucleotide coding sequence without SP |

TABLE 3
Sequence Listing

| | |
|---|---|
| 1 | ATKGTKRSYEQMETDGERQNATEIRASVGK |
| | MIDGIGRFYIQMCTELKLSDYEGRLIQNSL |
| | TIERMVLSAFDERRNKYLEEHPSAGKDPKK |
| | TGGPIYRRVDGKWRRELILYDKEEIRRIWR |
| | QANNGDDATAGLTHMMIWHSNLNDATYQRT |
| | RALVRTGMDPRMCSLMQGSTLPRRSGAAGA |
| | AVKGVGTMVMELIRMIKRGINDRNFWRGEN |
| | GRRTRIAYERMCNILKGKFQTAAQRTMVDQ |
| | VRESRNPGNAEFEDLIFLARSALILRGSVA |
| | HKSCLPACVYGSAVASGYDFEREGYSLVGI |
| | DPFRLLQNSQVYSLIRPNENPAHKSQLVWM |
| | ACHSAAFEDLRVSSFIRGTKVVPRGKLSTR |
| | GVQIASNENMETMESSTLELRSRYWAIRTR |
| | SGGNTNQQRASSGQISIQPTFSVQRNLPFD |
| | RPTIMAAFTGNTEGRTSDMRTEIIRLMESA |
| | RPEDVSFQGRGVFELSDEKATSPIVPSFDM |
| | SNEGSYFFGDNAEEYDN |
| 2 | KKQGDADVCGEVAYIQSVVSDCHVPTAELR |
| | TLLEIRKLFLEIQKLKVE |
| 3 | ZXBBBBZ |
| 4 | GRRRRRS |
| 5 | KKQGDADVCGEVAYIQSVVSDCHVPTAELR |
| | TLLEIRKLFLEIQKLKVEGRRRRRS |
| 6 | ATKGT |
| | KRSYEQMETDGERQNATEIRASVGKMIDGI |
| | GRFYIQMCTELKLSDYEGRLIQNSLTIERM |
| | VLSAFDERRNKYLEEHPSAGKDPKKTGGPI |
| | YRRVDGKWRRELILYDKEEIRRIWRQANNG |
| | DDATAGLTHMMIWHSNLNDATYQRTRALVR |
| | TGMDPRMCSLMQGSTLPRRSGAAGAAVKGV |
| | GTMVMELIRMIKRGINDRNFWRGENGRRTR |
| | IAYERMCNILKGKFQTAAQRTMVDQVRESR |
| | NPGNAEFEDLIFLARSALILRGSVAHKSCL |
| | PACVYGSAVASGYDFEREGYSLVGIDPFRL |
| | LQNSQVYSLIRPNENPAHKSQLVWMACHSA |
| | AFEDLRVSSFIRGTKVVPRGKLSTRGVQIA |
| | SNENMETMESSTLELRSRYWAIRTRSGGNT |
| | NQQRASSGQISIQPTFSVQRNLPFDRPTIM |
| | AAFTGNTEGRTSDMRTEIIRLMESARPEDV |
| | SFQGRGVFELSDEKATSPIVPSFDMSNEGS |
| | YFFGDNAEEYDNGSKKQGDADVCGEVAYIQ |
| | SVVSDCHVPTAELRTLLEIRKLFLEIQKLK |
| | VEGRRRRRS |
| 7 | GCGACTAAGGGCACGAAACGCAGCTACGAA |
| | CAAATGGAAACCGACGGTGAGCGTCAAAAT |
| | GCAACCGAAATCCGCGCTAGCGTCGGCAAG |
| | ATGATCGACGGCATCGGCCGTTTTTACATT |
| | CAGATGTGCACCGAGCTGAAGCTGAGCGAT |
| | TACGAGGGTCGTCTGATTCAGAATAGCTTG |
| | ACGATCGAGCGTATGGTGTTGAGCGCGTTC |
| | GATGAGCGCCGCAACAAATATCTGGAAGAA |
| | CATCCGAGCGCCGGTAAAGATCCGAAGAAA |
| | ACCGGTGGCCCTATCTACCGTCGTGTTGAT |
| | GGCAAGTGGCGTCGCGAGCTGATTCTGTAT |
| | GACAAAGAAGAAATTCGCCGTATTTGGCGC |
| | CAGGCGAATAATGGTGACGACGCGACCGCG |
| | GGTTTAACGCACATGATGATCTGGCATTCC |
| | AACCTGAACGATGCGACGTATCAACGTACC |
| | CGTGCGCTGGTGCGTACCGGCATGGACCCA |
| | CGTATGTGCTCGCTGATGCAAGGTTCCACC |
| | CTGCCTCGTCGTAGCGGTGCTGCCGGTGCG |
| | GCAGTGAAAGGTGTCGGCACGATGGTCATG |
| | GAACTTATCCGCATGATTAAGCGCGGTATC |
| | AATGATCGTAATTTCTGGCGCGGTGAGAAT |
| | GGTCGTCGTACCCGTATTGCGTATGAGCGT |
| | ATGTGCAACATTCTGAAGGGTAAATTCCAG |
| | ACCGCGGCACAGCGTACGATGGTCGACCAA |
| | GTTCGCGAGTCTCGTAACCCGGGCAATGCT |
| | GAGTTTGAAGATCTGATTTTCCTGGCGCGT |
| | AGCGCCCTGATTCTGCGTGGCTCGGTTGCG |
| | CACAAATCTTGTCTGCCGGCCTGCGTCTAT |
| | GGTAGCGCGGTGGCATCCGGTTACGACTTT |
| | GAGCGTGAGGGTTATAGCTTGGTTGGCATT |
| | GACCCGTTTCGCCTGCTGCAGAACAGCCAG |
| | GTGTACAGCCTGATCCGTCCAAATGAGAAC |
| | CCGGCACACAAGTCCCAACTGGTTTGGATG |
| | GCATGTCATAGCGCGGCTTTCGAAGATCTG |
| | CGTGTGTCTAGCTTTATCCGCGGTACCAAA |
| | GTTGTGCCGCGTGGCAAGCTGAGCACGCGT |
| | GGTGTGCAAATCGCCAGCAACGAAAACATG |
| | GAAACCATGGAATCTTCAACCCTGGAGCTG |
| | CGTAGCCGTTACTGGGCGATTCGCACCCGC |
| | AGCGGTGGCAATACCAACCAGCAACGTGCG |
| | AGCAGCGGCCAGATCAGCATTCAACCGACT |
| | TTTAGCGTTCAGCGTAATCTGCCGTTCGAC |
| | CGCCCGACGATCATGGCAGCCTTTACCGGT |
| | AACACCGAGGGTCGCACTAGCGACATGCGC |
| | ACCGAAATCATTCGCCTGATGGAGAGCGCC |
| | CGTCCGGAAGATGTCAGCTTCCAGGGTCGT |
| | GGTGTTTTCGAGCTGAGCGACGAGAAAGCG |
| | ACCTCCCCGATCGTCCCGAGCTTTGACATG |
| | TCTAACGAGGGCAGCTACTTTTTCGGTGAT |
| | AATGCAGAAGAGTACGATAAC |
| 8 | AAGAAACAGGGTGATGCTGACGTGTGCGGC |
| | GAAGTGGCATATATCCAGAGCGTCGTGAGC |
| | GATTGTCACGTTCCGACGGCAGAGTTGCGC |
| | ACGCTGTTGGAAATCCGTAAGCTGTTCTTG |
| | GAGATTCAAAAGCTCAAAGTTGAG |
| 9 | GGTCGTCGTCGCAGACGTTCC |
| 10 | AAGAAACAGGGTGATGCTGACGTGTGCGGC |
| | GAAGTGGCATATATCCAGAGCGTCGTGAGC |
| | GATTGTCACGTTCCGACGGCAGAGTTGCGC |
| | ACGCTGTTGGAAATCCGTAAGCTGTTCTTG |
| | GAGATTCAAAAGCTCAAAGTTGAGGGTCGT |
| | CGTCGCAGACGTTCC |
| 11 | GCGACTAAGGGCACGAAACGCAGCTACGAA |
| | CAAATGGAAACCGACGGTGAGCGTCAAAAT |
| | GCAACCGAAATCCGCGCTAGCGTCGGCAAG |
| | ATGATCGACGGCATCGGCCGTTTTTACATT |
| | CAGATGTGCACCGAGCTGAAGCTGAGCGAT |
| | TACGAGGGTCGTCTGATTCAGAATAGCTTG |
| | ACGATCGAGCGTATGGTGTTGAGCGCGTTC |
| | GATGAGCGCCGCAACAAATATCTGGAAGAA |
| | CATCCGAGCGCCGGTAAAGATCCGAAGAAA |
| | ACCGGTGGCCCTATCTACCGTCGTGTTGAT |
| | GGCAAGTGGCGTCGCGAGCTGATTCTGTAT |
| | GACAAAGAAGAAATTCGCCGTATTTGGCGC |
| | CAGGCGAATAATGGTGACGACGCGACCGCG |
| | GGTTTAACGCACATGATGATCTGGCATTCC |
| | AACCTGAACGATGCGACGTATCAACGTACC |
| | CGTGCGCTGGTGCGTACCGGCATGGACCCA |
| | CGTATGTGCTCGCTGATGCAAGGTTCCACC |
| | CTGCCTCGTCGTAGCGGTGCTGCCGGTGCG |
| | GCAGTGAAAGGTGTCGGCACGATGGTCATG |
| | GAACTTATCCGCATGATTAAGCGCGGTATC |
| | AATGATCGTAATTTCTGGCGCGGTGAGAAT |
| | GGTCGTCGTACCCGTATTGCGTATGAGCGT |
| | ATGTGCAACATTCTGAAGGGTAAATTCCAG |
| | ACCGCGGCACAGCGTACGATGGTCGACCAA |
| | GTTCGCGAGTCTCGTAACCCGGGCAATGCT |
| | GAGTTTGAAGATCTGATTTTCCTGGCGCGT |
| | AGCGCCCTGATTCTGCGTGGCTCGGTTGCG |
| | CACAAATCTTGTCTGCCGGCCTGCGTCTAT |
| | GGTAGCGCGGTGGCATCCGGTTACGACTTT |
| | GAGCGTGAGGGTTATAGCTTGGTTGGCATT |
| | GACCCGTTTCGCCTGCTGCAGAACAGCCAG |
| | GTGTACAGCCTGATCCGTCCAAATGAGAAC |
| | CCGGCACACAAGTCCCAACTGGTTTGGATG |
| | GCATGTCATAGCGCGGCTTTCGAAGATCTG |
| | CGTGTGTCTAGCTTTATCCGCGGTACCAAA |
| | GTTGTGCCGCGTGGCAAGCTGAGCACGCGT |
| | GGTGTGCAAATCGCCAGCAACGAAAACATG |
| | GAAACCATGGAATCTTCAACCCTGGAGCTG |
| | CGTAGCCGTTACTGGGCGATTCGCACCCGC |
| | AGCGGTGGCAATACCAACCAGCAACGTGCG |
| | AGCAGCGGCCAGATCAGCATTCAACCGACT |
| | TTTAGCGTTCAGCGTAATCTGCCGTTCGAC |
| | CGCCCGACGATCATGGCAGCCTTTACCGGT |
| | AACACCGAGGGTCGCACTAGCGACATGCGC |
| | ACCGAAATCATTCGCCTGATGGAGAGCGCC |
| | CGTCCGGAAGATGTCAGCTTCCAGGGTCGT |
| | GGTGTTTTCGAGCTGAGCGACGAGAAAGCG |
| | ACCTCCCCGATCGTCCCGAGCTTTGACATG |
| | TCTAACGAGGGCAGCTACTTTTTCGGTGAT |
| | AATGCAGAAGAGTACGATAACGGCAGCAAG |
| | AAACAGGGTGATGCTGACGTGTGCGGCGAA |
| | GTGGCATATATCCAGAGCGTCGTGAGCGAT |
| | TGTCACGTTCCGACGGCAGAGTTGCGCACG |
| | CTGTTGGAAATCCGTAAGCTGTTCTTGGAG |
| | ATTCAAAAGCTCAAAGTTGAGGGTCGTCGT |
| | CGCAGACGTTCC |

1. An A method for prophylaxis of influenza disease in a human subject in need thereof, said method comprising administering to said subject an immunogenic composition comprising: a fusion protein that
(i) comprises or essentially consists of SEQ ID NO:6, or
(ii) is a functional variant of SEQ ID NO:6 having at least 90% identity to SEQ ID NO: 6,
wherein said fusion protein is administered to said human subject in an amount of 180 μg, or more.

2. The method of claim 1, wherein an amount of 300 μg or more of said fusion protein is administered to said human subject.

3. (canceled)

4. (canceled)

5. (canceled)

6. (canceled)

7. (canceled)

8. (canceled)

9. (canceled)

10. (canceled)

11. The method of claim 1, wherein said fusion protein is administered via intramuscular route.

12. The method of claim 1, wherein said fusion protein is administered as a single injection to said human subject.

13. (canceled)

14. (canceled)

15. The method of claim 1, which provides total T-cell response specific to NP, CD4 T-cell response specific to NP, CD8 T-cell response specific to NP, or anti-NP IgG antibody response.

16. The method of claim 1, wherein an amount of 480 μg, or more, of said fusion protein is administered to said human subject.

17. The method of claim 1, which provides protection or cross-protection from influenza symptoms.

18. The method of claim 1, which provides protection or cross-protection from influenza symptoms of, influenza infection with influenza strain A or B.

19. The method of claim 1, wherein said fusion protein is administered at a dose of 180 μg to 1000 μg of said fusion protein.

20. The method of claim 1, wherein said fusion protein is administered at a dose of 300 μg or 480 μg.

21. The method of claim 1, wherein said immunogenic composition is administered in combination concomitantly or sequentially with a second immunogenic composition against influenza comprising one or more inactivated strains of influenza and/or an efficient amount of the hemagglutinin HA antigen from one or more influenza strains.

22. The method of claim 1, wherein one dose of an amount of 300 or 480 mg of said fusion protein is administered by intramuscular injection concomitantly with one dose of a second immunogenic composition comprising one or more inactivated strains of influenza or influenza hemagglutinin antigens.

23. The method of claim 1, wherein the composition does not comprise any adjuvant.

24. The method of claim 1, wherein the composition reduces the number of influenza like illnesses after 14 days of injection, with a dose of 300 mg or more, or 480 mg or more of said fusion protein, as compared to a placebo or to a dose of 90 mg.

25. The method of claim 1, which protects from severe influenza.

26. An immunogenic composition comprising
(i) a fusion protein of SEQ ID NO:6; or
(ii) a functional variant of SEQ ID NO:6 having at least 90% identity to SEQ ID NO:6;
at a concentration of 300 μg/mL or above,
and one or more pharmaceutically acceptable excipients.

27. The immunogenic composition of claim 26, wherein said fusion protein comprises at least 400 amino acid residues, for example between 400 and 600 amino acid residues, optionally, said fusion protein forms protein nanoparticles comprising nanoparticles with diameters of 20-100 nm and/or molecular weight of between 440-2200 kDa.

28. The immunogenic composition of claim 26, further comprising at least
i. a salt, e.g. sodium sulfate or sodium chloride, preferably sodium sulfate,
ii. an osmolyte, e.g. a sugar such as trehalose,
iii. a buffer, e.g. a phosphate buffer and/or a citrate buffer,
iv. optionally an antioxydant, e.g. methionine,
v. optionally a surfactant, e.g. polysorbate 80,
wherein the pH of the composition is between 6.0 and 7.0, typically between 6.3 and 6.6 and the osmolality is between 300 and 600 mOsm/kg, preferably between 400 and 500 mOsm/kg, for example about 450 mOsm/kg.

29. The immunogenic composition of claim 26, further comprising at least
i. sodium sulfate or sodium chloride,
ii. trehalose,
iii. a phosphate buffer and/or a citrate buffer,
iv. optionally methionine, and
v. optionally polysorbate 80,
wherein the pH of the composition is between 6.0 and 7.0, typically between 6.3 and 6.6 and the osmolality is between 300 and 600 mOsm/kg, preferably between 400 and 500 mOsm/kg, for example about 450 mOsm/kg.

30. The immunogenic composition of claim 26, which further comprises
i. sodium sulfate at a concentration of about 75 mM,
ii. trehalose at a concentration of about 200 mM,
iii. polysorbate 80 at a concentration between 0.02% and 0.08% (vol/vol), e.g. about 0.04%, and
iv. L-methionine at a concentration of about 5 mM.

31. The immunogenic composition of claim 26, wherein said composition does not comprise any adjuvant.

32. The immunogenic composition of claim 26, which is formulated as a ready-to-use sterile solution.

33. A method of prophylaxis for an influenza disease in a subject in need thereof, said method comprising administering an effective amount of an immunogenic composition of claim 26.

34. A method of inducing an antigen NP specific immune response in a subject, said method comprising administering an immunogenic composition comprising a fusion protein of SEQ ID NO:6, or a functional variant of SEQ ID NO:6 having at least 90% identity to SEQ ID NO:6, at a dose of 180 μg to 1000 μg of said fusion protein.

35. The method of claim 34, wherein said fusion protein is administered at a dose of 180 μg, 200 μg, 240 μg, 300 μg or 480 μg.
